(19) United States
(12) Patent Application Publication
(10) Pub. No.: US 2010/0330047 A1
(43) Pub. Date: Dec. 30, 2010

(54) Mesenchymal Stem Cells Grown Under Hypoxic Conditions: Compositions, Methods and Uses Therefor

(75) Inventor: Maria Giuditta Valorani, London (GB)

Correspondence Address: Zackson Law LLC, 1015 Locust Street Suite 750, St. Louis, MO, 63101-1324, US

(73) Assignee: LIFE & LIGHT LTD., London (GB)

(21) Appl. No.: 12/825,201

(22) Filed: Jun. 28, 2010

Related U.S. Application Data
(60) Provisional application No. 61/269,603, filed on Jun. 26, 2009.

(51) Int. Cl. A61K 35/12; C12N 5/07; C12N 5/071; C12N 5/02; A61P 17/02; A61P 9/00

(52) U.S. Cl. 424/93.7; 435/352; 435/366; 435/374; 435/375; 435/383

(57) ABSTRACT

Methods of forming ex vivo cell cultures comprising differentiated mesenchymal lineage cells are disclosed. These methods comprise a) providing a cell culture comprising a plurality of mesenchymal stem cells (MSCs); b) subjecting the MSCs to hypoxic conditions; and c) subsequent to b), subjecting the MSCs to normoxic conditions. Enhanced differentiation of various mesenchymal lineage cells can be achieved for mammalian cells such as murine cells or human cells.

### PRIORITY

The present application claims the benefit of priority of U.S. Provisional Application 61/269,603 filed Jun. 26, 2009, which is incorporated herein by reference in its entirety.

### FIELD

The present disclosure generally relates to mesenchymal stem cell (MSC) cultures such as adipose tissue MSCs and bone marrow MSCs, in which the cells are exposed to hypoxic conditions ex vivo. The present disclosure also generally relates to uses of such cell cultures.

### INTRODUCTION

Oxygen status is an important factor influencing all major aspects of cell biology including survival, proliferation, differentiation, and migration. Mammalian cells require a constant supply of oxygen to maintain adequate energy production, and to ensure normal cell function and cell survival. However, it is known that stem cells in the bone marrow reside in a hypoxic environment (with oxygen tension ranging from 1% to 7%) (Hung et al., 2007). This hypoxic environment is required for maintaining bone marrow stem cells' proliferation and self-renewal capability (Ivanovic, 2000; Ivanovic, 2000). Several recent studies have investigated the effects of reduced oxygen tension on rat, murine and human mesenchymal stem cells (MSCs) derived both from bone marrow (BM) and adipose tissue (AT) (Ren, 2006; Potier, 2007; Malladi 2006). Additionally, it has been noted that short-term culture of MSCs under hypoxic conditions may provide a general method of enhancing their engraftment in vivo into a variety of tissues (Hung et al., 2007). Adipose derived MSCs are deemed more advantageous as a cell source than mature adipocytes (Sterodimas, A., et al., J. Plast. Reconstr. Aesthet. Surg. 62: 447-452, 2009; Cherubino, M., et al., Regen. Med. 4: 109-117, 2009; Yoshimura, K., et al., Regen. Med. 4: 265-273, 2009). Mature adipocytes may not be the best source of cells for tissue regeneration because they have already differentiated and committed to a specific cell type (Gomillion, C. and Burg, K., Biomaterials 27: 6052-6063, 2006).

Fink, T. et al., Stem Cells 22:1346-1355, 2004 used an immortalized human cell line, the hMSC-TERT cell line derived from human bone marrow stromal cells to show that these transformed cells, when incubated under hypoxic conditions (1% oxygen), form an adipocyte-like phenotype with cytoplasmic accumulation of lipid. However, in spite of increased levels of the PPAR-γ-induced angiopoietin-related gene (PGAR) transcripts, the accumulation of lipids was not accompanied by increased transcription of adipocyte-specific genes such as ADD1/SREBP1c, PPAR-γ2, lipoprotein lipase, aP2, leptin, perilipin, or adipophilin. Hence, these cells acquired an adipocyte-mimicking morphology in the absence of true adipogenic conversion.

Culturing human adipose-derived mesenchymal stem cells (hAT-MSCs) under hypoxia conditions induces cellular and molecular changes and can enhance their skin-regenerative potential through up regulating secretion of growth factors and through effects on functions such as angiogenesis, anti-apoptosis and wound healing (Chung, H. M., et al., Expert Opin Biol Ther. 9: 1499-1508,2009). Adipose tissue in vocal fold lipoinjection is currently used to treat patients affected by laryngeal hemiplegia or anatomical defects (Lo Cicero, V., et al., Cell Prolif. 41: 460-473, 2008.)

### SUMMARY

The present inventor has realized that better methods and better cell cultures are needed for providing mesenchymal stem cells from primary sources rather than transformed cell lines, in undifferentiated or in differentiated states, in sufficient amounts of cells and in sufficient purity. Such cells can be used for various medical purposes. Accordingly, the inventor has developed methods of producing ex vivo cell cultures comprising differentiated mesenchymal lineage cells. The ex vivo cell cultures and methods of forming such cultures set forth herein can provide, in various embodiments, greater numbers and percentages of cells that can proliferate as mesenchymal stem cells and/or can differentiate into one or more mesenchymal lineages, such as adipose lineage cells, chondrocyte lineage cells and/or osteogenic lineage cells. Furthermore, the present techniques utilize primary cells, rather than cells that derive from transformed or immortalized (and potentially tumorogenic) cell lines. Primary cells of the various embodiments can be of human origin, murine origin, avian cells, or originate from any other vertebrate species.

Accordingly, the inventor discloses herein methods of forming ex vivo cell cultures comprising differentiated mesenchymal lineage cells. In various aspects, these methods can comprise: providing a cell culture comprising a plurality of mesenchymal stem cells (MSCs), and subjecting the MSCs to hypoxic conditions. In further aspects, the methods can comprise subjecting the MSCs to normoxic conditions subsequent to culture under hypoxic conditions. In various aspects, culturing MSCs using the disclosed methods can enhance MSC production, enrichment and adipogenic differentiation.

In various embodiments of the present teachings, MSCs can be adipose tissue MSCs (AT-MSCs), such as, without limitation, epiploon AT-MSCs. In other embodiments, MSCs can be bone marrow MSCs (BM-MSCs). In other embodiments, MSCs can be testis tissue MSCs (TT-MSCs). In yet other embodiments, MSCs can be pancreas-derived MSCs (P-MSCs). In some configurations, AT-MSCs can be obtained from omental fat. In some configurations, AT-MSCs can be selected for their ability to attach to a plastic substratum such as cell culture plastic, and can be grown under normoxic and hypoxic conditions. In some embodiments, the methods can involve prior exposure of MSCs to hypoxia, which can lead to a reduction of ex vivo expansion time, and can also lead to increased numbers of Sca-1⁺ as well as Sca-1⁺/CD44⁺double-positive cells compared to controls. In various configurations of the methods, under low oxygen culture conditions, the AT-MSC number can increase, and their adipogenic differentiation potential can be reduced, compared to controls. Notably, the hypoxia-mediated inhibition of adipogenic differentiation was reversible: AT-MSCs pre-exposed to hypoxia when switched to normoxic conditions exhibited significantly higher adipogenic differentiation capacity compared to their pre-exposed normoxic-cultured counterparts. Accordingly, in some configurations of the methods, the expression of adipocyte-specific genes, peroxisome proliferator activated receptor γ (Pparγ), lipoprotein lipase (Lpl) and fatty acid binding protein 4 (Fabp4) can be significantly enhanced in hypoxia pre-exposed AT-MSCs.

In various configurations of the methods, subjecting MSCs to hypoxic conditions can comprise subjecting the MSCs to an atmosphere comprising less than 21% oxygen, such as an atmosphere comprising no more than about 10% oxygen, such as an atmosphere comprising from 0.2% oxygen up to 10% oxygen, or from about 1% oxygen up to about 10% oxygen. In further configurations of the methods, subjecting MSCs to hypoxic conditions can comprise subjecting the MSCs to an atmosphere comprising no more than about 7% oxygen, such as an atmosphere comprising from 0.1% oxygen up to 7% oxygen, 0.2% oxygen up to 7% oxygen, or 1% oxygen up to 7% oxygen. In various embodiments of the methods, subjecting the MSCs to hypoxic conditions can comprise subjecting the MSCs to an atmosphere comprising no more than about 2% oxygen. In further embodiments of the methods, the subjecting the MSCs to hypoxic conditions can comprise subjecting the MSCs to an atmosphere comprising no more than 2% oxygen, no more than 3% oxygen, no more than 4% oxygen, or no more than 5% oxygen. In further configurations of the methods, the atmosphere can further comprise about 5% CO₂.

In various configurations of the methods, MSCs can be subjected to culture under hypoxic conditions for any suitable duration, such as from 1 day up to 100 days, from 1 day up to 90 days, from 3 days up to 21 days, or from 8 days up to 14 days. In additional configurations, MSCs can be subjected to hypoxic conditions for from 8 days up to 11 days, or from 9 days up to 11 days. In yet other configurations, subjecting the cells to hypoxic conditions can comprise subjecting the cells to hypoxic conditions for about 10 days.

In additional embodiments of the methods, differentiated mesenchymal cells such as differentiated AT-MSCs can include adipocytes. In some configurations, a cell culture can comprise at least 78% adipocyte lineage cells. In some configurations, a cell culture can comprise at least 79% adipocyte lineage cells. In some configurations, a cell culture can comprise at least 80% adipocyte lineage cells. In some configurations, a cell culture can comprise at least 81% adipocyte lineage cells. In other embodiments, differentiated mesenchymal cells such as differentiated AT-MSCs can include osteocytic lineage cells. In further embodiments, differentiated mesenchymal cells such as differentiated AT-MSCs can include chondrogenic lineage cells. In some aspects, an ex vivo cell culture exposed to hypoxic conditions can comprise an enhanced percentage of Oil Red O-staining cells compared to a control culture exposed to normoxic conditions. In other aspects, an ex vivo cell culture pre-exposed to hypoxic conditions can comprise an enhanced percentage of Alcian Blue-staining cells compared to a control culture pre-exposed to normoxic conditions. In other aspects, an ex vivo cell culture pre-exposed to hypoxic conditions can include an enhanced percentage of Von Kossa-staining cells compared to a control culture pre-exposed to normoxic conditions. In additional aspects, an ex vivo cell culture can further include a medium comprising hydrocortisone, isobutyl methyl xanthine, indomethacin, insulin or a combination thereof, in amounts effective for adipogenic differentiation. In yet other aspects, an ex vivo cell culture can further comprise a medium comprising basic Fibroblast Growth Factor (bFGF), Transforming Growth Factor-β1 (TGF β1), or a combination thereof, in amounts effective for chondrogenic differentiation. In further aspects, an ex vivo cell culture can further comprise a medium comprising dexamethasone, vitamin C phosphate, sodium β-glycerophosphate, or a combination thereof, in amounts effective for osteogenic differentiation.

In other aspects of the present teachings, an ex vivo cell culture can comprise a tissue comprising mesenchymal stem cells subjected to hypoxic conditions ex vivo as described herein. In some configurations, a tissue can be adipose tissue, osteocytic tissue, or chondrogenic tissue.

In other aspects of the present teachings, the inventor discloses methods of repairing or augmenting a tissue or organ in a subject. In various configurations, these methods can comprise: providing a cell culture comprising a plurality of mesenchymal stem cells (MSCs), subjecting the MSCs to hypoxic conditions, and transplanting cells comprised by the cell culture to the subject. In some aspects, the methods can also comprise subjecting the MSCs to normoxic conditions subsequent to the hypoxic conditions. In some configurations, cells of the present teachings that can be used in repairing or augmenting a tissue or organ can be cells that are autologous to a subject. In various configurations, the differentiated cells can be, without limitation, adipocyte lineage cells, osteocytic lineage cells, chondrogenic lineage cells or a combination thereof. In other configurations, a tissue or organ can be, without limitation, a tissue or organ such as breast (Yoshikawa T., Plast. Reconstr. Surg. 121: 860-877, 2008) cheek, chin, lips, heart (Hu, X., J. Thorac. Cardiovasc. Surg. 135: 799-808, 2008), vasculature, adipose tissue, vocal folds (Lo Cicero, V., et al., Cell Prolif. 41, 460-473, 2008), an intervertebral disc (Kanichai, M., et al., J. Cell Physiol. 216: 708-715, 2008), stomach (e.g., gastric ulcer treatment, Wu, Y., et al., Stem Cells, 25: 2648-2659, 2007) or pancreas (e.g., beta cell) deficiency (Timper, K. et al., Biophys. Biochem. Res. Comm. 341: 1135-1140, 2006).

In some configurations, the present methods can be used to enhance the paracrine effects of MSCs (Gnecchi, M., et al., FASEB J. 20: 661-669, 2006).

In additional aspects, methods of the present teachings include methods of growing mesenchymal stem cells (MSCs) ex vivo. In various configurations, these methods can comprise: providing a culture comprising MSCs; and subjecting the culture to hypoxic conditions, wherein the MSCs express at least one marker of MSC differentiation in an amount greater than that of a control culture comprising MSCs subjected to normoxic conditions. In some embodiments, the at least one marker of MSC differentiation is selected from the group consisting of Sca 1 and CD44. In other configurations of the methods, a greater percentage of cells express Sca 1 and CD44 compared to a control comprising MSCs subjected to normoxic conditions. In further configurations of the methods, the MSCs can express the at least one marker of MSC differentiation in a greater percentage of cells compared to a control culture comprising MSCs subjected to normoxic conditions. In additional embodiments of the methods, the MSCs can be adipose tissue MSCs (AT-MSCs). In further embodiments, the MSCs are bone marrow MSCs (BM-MSCs).

The present inventor sets forth herein methods of forming an ex vivo cell culture. In various embodiments, these methods can comprise: providing adipose tissue mesenchymal stem cells; and growing the cells under hypoxic conditions. In various configurations, the cells can express one or more genes involved in adipogenesis differentiation at a level at least two-fold greater than a control cell culture that is subjected to normoxic conditions. In various aspects of these methods, an adipocyte lineage differentiation gene can be PPARγ, LPL or FBP4. In further aspects, mesenchymal stem cells grown under hypoxic conditions can exhibit an accumulation of lipids greater than that exhibited by control cells grown under normoxic conditions. In yet other aspects, the accumulation of lipids may or may not be accompanied by cells grown under hypoxic conditions. These cells can exhibit increased transcription of adipocyte-specific genes such as ADD1/SREBP1c, PPAR-γ2, lipoprotein lipase, aP2, leptin, perilipin, and adipophilin in comparison to controls grown under normoxic conditions.

In additional aspects, the present teachings also include methods of increasing proliferation rate of a cell culture ex vivo. In some embodiments, the methods can comprise growing the cells ex vivo under hypoxic conditions. In further embodiments the methods can comprise growing the cells under hypoxic conditions, wherein the proliferation rate of the hypoxic cell culture is greater than that of a control cell culture grown under normoxic conditions. In additional embodiments of the methods, the culture can comprise stem cells. In further embodiments, the stem cells are mesenchymal stem cells (MSCs). In some embodiments of the methods, the mesenchymal stem cells are adipose tissue mesenchymal stem cells (AT-MSCs). In other embodiments, the mesenchymal stem cells are bone marrow mesenchymal stem cells (BM-MSCs). In other embodiments, the mesenchymal stem cells can be pancreas-derived mesenchymal stem cells or testis tissue mesenchymal stem cells (P-MSCs or TT-MSCs, respectively).

In additional aspects, the present teachings also provide methods of enhancing expression of at least one pluripotent stem cell marker in an ex vivo cell culture. In some embodiments, the methods can comprise providing a cell culture comprising a plurality of mesenchymal stem cells (MSCs); and subjecting the MSCs to hypoxic conditions, wherein a greater percentage of cells express the at least one pluripotent stem cell marker compared to a cell culture comprising cells subjected to normoxic conditions. In some embodiments of the methods, the plurality of MSCs is a plurality of adipose tissue mesenchymal stem cells (AT-MSCs). In other embodiments of the methods, the plurality of MSCs is a plurality of bone marrow mesenchymal stem cells (BM-MSCs). In additional embodiments of the methods, the pleuripotent stem cell marker is selected from the group consisting of Sca1 and CD44. In some embodiments of the methods, greater than 35% of the MSCs are enriched in Sca1 and CD44. In further embodiments of the methods, greater than 35% up to about 80% of the AT-MSCs are enriched in Sca1 and CD44.

In further aspects, the present teachings disclose methods of maintaining mesenchymal stem cells in an undifferentiated state in culture. In some embodiments, these methods comprise maintaining the mesenchymal stem cells under hypoxic conditions ex vivo. In some embodiments, the methods can comprise maintaining the mesenchymal stem cells in an atmosphere comprising no more than 10% oxygen, such as, 0.1% oxygen to 10% oxygen, 0.2% oxygen to 10% oxygen, or 1% oxygen to 10% oxygen. In other embodiments, a method can comprise maintaining the mesenchymal stem cells in an atmosphere comprising from 0.2% to 3% oxygen, or from 1% oxygen up to 3% oxygen. In some embodiments, a method can comprise maintaining the mesenchymal stem cells in an atmosphere comprising about 2% oxygen. In some embodiments, a method can comprise maintaining the mesenchymal stem cells in an atmosphere comprising 2% oxygen.

In additional aspects, the present inventor describes methods of enhancing expression of at least one adipogenic lineage gene in an ex vivo cell culture. In some configurations, the methods comprise providing an ex vivo cell culture comprising mesenchymal stem cells (MSCs) and growing the cells under hypoxic conditions. In some aspects, the methods involve returning the cells to normoxic conditions, whereby the at least one adipogenic lineage genes is expressed at a level greater than that of a control culture grown under normoxic conditions. In some embodiments of the methods, the mesenchymal stem cells are adipose tissue mesenchymal stem cells (AT-MSCs). In some embodiments, the adiopogenic lineage genes are selected from the group consisting of PPARγ, LPL and FABP.

In some aspects, the methods disclosed herein can be used to enhance human adipose-derived mesenchymal stem cells (hAT-MSCs) differentiation in vitro into the adipogenic lineage. Cells grown under the disclosed conditions can be used, for example, in plastic and reconstructive surgery and in tissue engineering, such as, for example, in therapies performed after oncological resections and complex traumas or augmentative surgery of the breast, cheek, chin or lips.

In alternative aspects, the present description discloses methods of promoting healing of a gastric ulcer. In additional aspects, the method comprises forming an ex vivo cell culture comprising differentiated adipose tissue MSCs. In these aspects, subjecting the MSCs to normoxic conditions comprises subjecting the MSCs to normoxia under conditions that promote expression of mRNAs for VEGF and hepatocyte growth factor (HGF). In further embodiments, the methods comprise transplanting the cells to gastric tissue surrounding the ulcer in a subject in need of treatment. (Hayashi, Y. et al., Am. J. Physiol. Gastrointest. Liver. Physiol. 294: G778-G786, 2008.)

In additional aspects, the present inventor describes methods of promoting heart regeneration in a subject. In some embodiments, these methods can comprise forming a cell culture comprising differentiated adipose tissue mesenchymal stem cells (AT-MSCs) grown under hypoxic conditions ex vivo. In additional embodiments these methods further comprise subjecting the MSCs to normoxic conditions that promote increased expression of pro-survival and pro-angiogenic factors. In further embodiments, these methods can comprise transplanting the cells to a diseased area of the heart in a subject in need of treatment. (Hu, X., et al., J. Thorac. Cariovasc. Surg. 135: 799-808, 2008.)

In some aspects, the inventor discloses methods of promoting wound healing in a subject. In sonic embodiments, the methods comprise forming an ex vivo cell culture comprising differentiated adipose tissue mesenchymal stem cells (AT-MSCs) that have been subjected to hypoxic conditions. In additional embodiments, these methods can comprise transferring to normoxic conditions the AT-MSCs that have been subjected to hypoxic conditions. In some configurations, the AT-MSCs can be subjected to normoxia under conditions that promote increased expression and/or release of proangiogenic factors (Wu, Y., et al., Stem Cells 25: 2648-2659, 2007). In further aspects, the methods can comprise transplanting the cells to a wound, to diseased tissue, or to the area near a wound or diseased tissue, such as an area of a diseased heart in a subject in need of treatment. For example, the present methods can be used in various embodiments for cutaneous regeneration and wound healing through differentiation and paracrine effects (Wu, Y., et al., Stem Cells 25: 2648-2659, 2007).

In further aspects, the inventor discloses methods of promoting repair, expansion, augmentation or regeneration of a tissue in a subject. In some embodiments, the methods comprise forming an ex vivo cell culture comprising differentiated adipose tissue mesenchymal stem cells (AT-MSCs). In further embodiments, the subjecting the MSCs to normoxic conditions comprises subjecting the MSCs to normoxia under conditions that promote increased expression of pro-survival and pro-angiogenic factors. In additional embodiments, the methods comprise transplanting the cells to a diseased area of the tissue in a subject in need of treatment. (Hu, X., et al., J. Thorac. Cariovasc. Surg. 135: 799-808, 2008.) In some embodiments of the methods, the tissue which can be repaired, expanded, augmented or regenerated using the disclosed methods can be, without limitation, breast, cheek, chin, lip or vocal fold.

In additional aspects, the inventor discloses ex vivo cell cultures comprising mesenchymal stem cells differentiated as adipose lineage cells. In some aspects, the mesenchymal stem cells are differentiated at a greater percentage compared to a control ex vivo cell culture comprising adipose tissue mesenchymal stem cells pre-grown under normoxic conditions. In further embodiments, the adipose lineage cells can include, without limitation, adipocytes, osteocytes, chondrocytes or combination thereof.

In additional embodiments, a cell culture can comprise a plurality of adipocytes. In these embodiments, a cell culture can further comprise hydrocortisone, isobutyl xanthine, indomethacin and insulin. In other embodiments, a culture can comprise a plurality of chondrocytes. In these embodiments, a culture can further comprise basic Fibroblast Growth Factor and Transforming Growth Factor-β1. In still other embodiments, a culture can comprise a plurality of osteocytes. In these embodiments, a culture can further comprises dexamethasone, vitamin C phosphate, and sodium-β-glycerophosphate.

### BRIEF DESCRIPTION OF THE DRAWINGS

The drawings described below are for illustrative purposes only and are not intended to limit the scope of the present teachings in any way.

FIG. 1: This figure illustrates effect of hypoxia on murine BM-MSCs at 90 days.

FIG. 2 illustrates differentiation potential of normoxic cultured murine AT-MSC.

FIG. 3 illustrates effect of hypoxia on the expression of stem cell markers in murine AT-MSC.

FIG. 4 illustrates effect of hypoxia on murine cell growth, survival and cell cycle distribution.

FIG. 5 illustrates that hypoxia inhibits murine AT-MSC adipogenic differentiation.

FIG. 6 illustrates that pre-hypoxic-cultured murine AT-MSCs display enhanced adipogenic differentiation potential when exposed to normoxia.

FIG. 7 illustrates that low oxygen levels enhances the number of Sca-1⁺/CD44⁺ cells in the MSC fractions obtained from both pancreas and testis.

FIG. 8 illustrates that both hypoxic and normoxic murine cells exhibit a small, spindle-shaped morphology.

FIG. 9 illustrates enhanced adipogenic differentiation pre-hypoxic conditions. These cells are AT-MSCs from liposuction of human donor 20 year old female.

FIG. 10 illustrates assessment of adipogenic differentiation in hAT-MSCs in cells of the donor in FIG. 9. The number of adipocytes were counted on a phase contrast microscope (n=4).

FIG. 11 illustrates pre-hypoxic-cultured hAT-MSCs from a second donor display enhanced adipogenic differentiation potential when exposed to normoxia. These cells are AT-MSCs from liposuction of human donor 23 year old female.

FIG. 12 illustrates assessment of adipogenic differentiation in hAT-MSCs from the donor of FIG. 11. The number of adipocytes were counted on phase contrast microscope (n=4).

FIG. 13 illustrates pre-hypoxic-cultured hAT-MSCs from a third donor display enhanced adipogenic differentiation potential when exposed to normoxia. These cells are AT-MSCs from liposuction of human donor 55 year old female.

FIG. 14 illustrates assessment of adipogenic differentiation in hAT-MSCs from the donor of FIG. 13. The number of adipocytes were counted on phase contrast microscope (n=4).

### DETAILED DESCRIPTION

The present teaching discloses that bone marrow (BM) and adipose tissue (AT) cells can be a source of pure MSCs.

The present inventor has shown that MSCs that have been exposed to hypoxic conditions can have enhanced expression of pluripotent stem cell markers such as CD44 and Sca-1. The present inventor has further demonstrated that prior exposure of MSCs to hypoxic culture conditions can be used in methods of enhancing MSC production and purification and for increasing the stem cell pool. The present inventor has found that pre-hypoxia exposure can enhance proliferation, can protect from death, and can inhibit adipogenic differentiation of AT-MSCs. Under this condition, re-oxygenation can potentiate the differentiation ability of these cells into adipocytes. The present inventor has also demonstrated that subsequent exposure to hypoxic culture conditions can enhance the cells' differentiation potential compared to normoxic-cultured MSCs.

As described herein, Non Obese Diabetic (NOD) mice, a model of Type 1 diabetes, were used as a source of pure MSCs. Such MSCs were expanded and enriched at low (about 2%) and normal oxygen levels. The capacity of prior normoxia-/hypoxia-cultured AT-MSCs to differentiate in vitro into the adipogenic lineage was analyzed by quantifying the expression of adipogenic genes in the MSCs and/or differentiated cells.

Mesenchymal Stem Cells

Described herein are methods of culturing of mesenchymal stem cells so as to provide differentiated cells of various mesenchymal lineages. Except as otherwise provided herein, such cells can be isolated, purified, or cultured by any of a variety of methods known in the art (e.g., Vunjak-Novakovic and Freshney (2006) Culture of Cells for Tissue Engineering, Wiley-Liss, ISBN-10 0471629359; Challen and Little (2006) Stem Cells 24(1), 3-12; Lanza et al., eds. (2004) Handbook of Stem Cells, Academic Press, ISBN 0124366430; Lanza et al., eds. (2005) Essentials of Stem Cell Biology, Academic Press, ISBN 0120884429; Saltzman (2004) Tissue Engineering: Engineering Principles for the Design of Replacement Organs and Tissues, Oxford ISBN 019514130X; Minuth et al. (2005) Tissue Engineering: From Cell Biology to Artificial Organs, John Wiley & Sons, ISBN 3527311866). Such methods can be utilized directly or adapted for use with the methods described herein.

Mesenchymal stem cells of the present teachings can be derived from the same or different species as a transplant recipient. For example, mesenchymal stem cells can be derived from an animal, including, but not limited to, a mammal or an avian, such as a human, a horse, a cow, a companion animal such as a dog or a cat, an agricultural animal such as a sheep, a pig, a chicken, or a laboratory animal such as a rodent, for example a mouse, a rat or a guinea pig. The mesenchymal stem cells can be derived from the transplant recipient or from another subject of the same or different species. In some configurations, mesenchymal stem cells of the present teachings can be of mammalian origin other than murine mesenschymal stem cells, and can be, for example, human mesenchymal stem cells.

In various aspects of the present teachings, a mesenchymal stem cell can be a progenitor cell capable of growth ex vivo. In other aspects, mesenchymal stem cells can differentiate into cells of a tissue or organ, such as, for example, osteoblasts, chondrocytes, myocytes, adipocytes, neuronal cells, and/or beta-pancreatic islets cells. In some aspects, a mesenchymal stem cell can be an undifferentiated stem cell.

In some embodiments, MSCs of the present teachings can be adipose tissue MSCs (AT-MSCs), such as, for example, epiploon AT-MSCs. In some embodiments, the MSCs can be bone marrow MSCs (BM-MSCs). In some embodiments, the MSCs can be pancreatic MSCs (P-MSCs). In some embodiment, the MSCs can be testis tissue MSCs (TT-MSCs).

In some embodiments, a mesenchymal stem cell can comprise a heterologous nucleic acid so as to express a bioactive molecule, or heterologous protein or to overexpress an endogenous protein. As an example, the mesenchymal stem cell to be cultured can be genetically modified to expresses a fluorescent protein marker. Exemplary markers include GFP, EGFP, BFP, CFP, YFP, and RFP (Chalfie, M. and Kain, S., Green Fluorescent Protein Properties, Applications, and Protocols, Second Edition. John Wiley and Sons, 2005. ISBN 0471736821, 9780471736820; Serdyuk, l.g., et al., Methods in Molecular Biophysics, Cambridge University Press, ISBN 052181524X, 9780521815246, 2007). As another example, a mesenchymal stem cell can be a genetically modified MSC that expresses or up-regulates expression of a polypeptide, such as, for example, an angiogenesis-related factor, such as activin A, adrenomedullin, aFGF, ALK1, ALK5, ANF, angiogenin, angiopoietin-1, angiopoietin-2, angiopoietin-3, angiopoietin-4, angiostatin, angiotropin, angiotensin-2, AtT20-ECGF, betacellulin, bFGF, B61, bFGF inducing activity, cadherins, CAM-RF, cGMP analogs, ChDI, CLAF, claudins, collagen, collagen receptors α₁β₁ and α₂β₁, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TNF-alpha, TGF-beta, PD-ECGF, PDGF, IGF, IL8, growth hormone, fibrin fragment E, FGF-5, fibronectin and fibronectin receptor α5β1, Factor X, HB-EGF, HBNF, HGF, HUAF, heart derived inhibitor of vascular cell proliferation, IFN-gamma, IL1, IGF-2 IFN-gamma, integrin receptors, K-FGF, LIF, leiomyoma-derived growth factor, MCP-1, macrophage-derived growth factor, monocyte-derived growth factor, MD-ECI, MECIF, MMP 2, MMP3, MMP9, urokiase plasminogen activator, neuropilin (NRP1, NRP2), neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (SIP1), Syk, SLP76, tachykinins, TGF-beta, Tie 1, Tie2, TGF-β, and TGF-β receptors, TIMPs, TNF-alpha, TNF-beta, transferrin, thrombospondin, urokinase, VEGF-A, VEGF-B, VEGF-C, VEGF-D, VEGF-E, VEGF, VEGF.sub.164, VEGI, EG-VEGF, VEGF receptors, PF4, 16 kDa fragment of prolactin, prostaglandins E1 and E2, steroids, heparin, 1-butyryl glycerol (monobutyrin), and/or nicotinic amide. As another example, a mesenchymal stem cell can comprise a genetic modification that renders the cell capable of reducing or eliminating an immune response in the host (e.g., through down-regulation of expression of a cell surface antigen such as class I and class II histocompatibility antigens).

In some embodiments, a mesenchymal stem cell can be cultured with one or more cell types in addition to a first mesenchymal stem cell. Such additional cell types can include (but are not limited to) skin cells, liver cells, heart cells, kidney cells, pancreatic cells, lung cells, bladder cells, stomach cells, intestinal cells, cells of the urogenital tract, breast cells, skeletal muscle cells, skin cells, bone cells, cartilage cells, keratinocytes, hepatocytes, gastro-intestinal cells, epithelial cells, endothelial cells, mammary cells, skeletal muscle cells, smooth muscle cells, parenchymal cells, osteoclasts, or chondrocytes. These cell types can be introduced prior to, during, or after culture of a mesenchymal stem cell. Such introduction can take place in vitro or in vivo. When the cells are introduced in vivo, the introduction can be at the tissue or organ transplant site or at a site removed therefrom. Exemplary routes of administration of the cells include injection and surgical implantation.

Differentiated Mesenchymal Lineage Cells

In various methods described herein, mesenchymal stem cells can be cultured under hypoxic conditions so as to result in a differentiated cell line. Differentiated cell lines produced according to methods described herein include, but are not limited to, osteoblasts, chondrocytes, myocytes, adipocytes, neuronal cells, and beta-pancreatic islets cells. For example, differentiated cell lines produced according to methods described herein include, but are not limited to, skin cells, liver cells, heart cells, kidney cells, pancreatic cells, lung cells, bladder cells, stomach cells, intestinal cells, cells of the urogenital tract, breast cells, skeletal muscle cells, skin cells, bone cells, cartilage cells, keratinocytes, hepatocytes, gastro-intestinal cells, epithelial cells, endothelial cells, mammary cells, skeletal muscle cells, smooth muscle cells, parenchymal cells, osteoclasts, or chondrocytes.

In some embodiments, a differentiated cell line can comprise adipocytes. For example, according to protocols described herein, mesenchymal stem cells pre-cultured under hypoxic conditions can comprise at least 80% adipocyte lineage cells in the culture.

In some embodiments, a differentiated cell line can comprise osteocytic lineage cells. In some embodiments, a differentiated cell line can comprise chondrogenic lineage cells.

MSC Differentiation Markers

In various methods described herein, mesenchymal stem cells can be grown ex vivo under hypoxic conditions so as to result in MSCs that express at least one marker of MSC differentiation. For example, hypoxic culture of mesenchymal stem cells can result in MSCs that express at least one marker of MSC differentiation in an amount greater than that of a control culture comprising MSCs subjected to normoxic conditions.

Markers of MSC differentiation include, but are not limited to Sca1 and CD44. As an example, hypoxic culture of mesenchymal stem cells can result in a greater percentage of cells that express Sca1 or CD44 compared to a control comprising MSCs subjected to normoxic conditions.

In some embodiments, hypoxic culture of mesenchymal stem cells can result in MSCs that express elevated levels of adipocyte lineage differentiation markers. For example, adipocyte lineage differentiation markers include, but are not limited to, PPARγ, LPL and FBP4. Under various hypoxic culture protocols described herein, an ex vivo cell culture can express one or more adipogenic markers at a level at least two-fold greater than a control cell culture that is subjected to normoxic conditions.

In some embodiments, hypoxic culture of mesenchymal stem cells can result in MSCs that express elevated levels of markers of bone marrow MSCs (BM-MSCs).

In some methods of the present teachings, hypoxic culture of mesenchymal stem cells can enhance expression of at least one pluripotent stem cell marker in an ex vivo cell culture, in comparison to a control normoxic culture. Various protocols for hypoxic culture of mesenchymal stem cells described herein can result in greater percentage of cells that express at least one pluripotent stem cell marker compared to a cell culture comprising cells subjected to normoxic conditions. Pluripotent stem cell markers include, but are not limited to, Sca1 and CD44. For example, hypoxic culture of mesenchymal stem cells can result in a culture in which greater than 35% of the MSCs are enriched in Sca1 and/or CD44. As another example, hypoxic culture of mesenchymal stem cells can result in a culture in which greater than 35% up to about 80% of the AT-MSCs are enriched for accumulation of Sca1 and/or CD44. In some embodiments, adipose tissue mesenchymal stem cells (AT-MSCs) or bone marrow mesenchymal stem cells (BM-MSCs) can be cultured under hypoxic conditions to enhance expression of at least one pluripotent stem cell marker in an ex vivo cell culture. For example, a cell culture comprising AT-MSCs subjected to hypoxic conditions can result in a greater percentage of cells that express at least one pluripotent stem cell marker compared to a cell culture comprising AT-MSCs subjected to normoxic conditions.

Hypoxic Conditions

As described herein, culture of mesenchymal stem cells under hypoxic conditions can, inter aria, result in increased differentiation of a mesenchymal stem cell line and increase markers of MSC differentiation.

Hypoxic conditions can include a level of oxygen lower than those of conventional culture conditions.

According to methods described herein, hypoxic conditions can comprise an oxygen level of lower than 10%. In some embodiments, hypoxic conditions comprise up to about 7% oxygen. For example, hypoxic conditions can comprise up to about 7%, up to about 6%, up to about 5%, up to about 4%, up to about 3%, up to about 2%, or up to about 1% oxygen. As another example, hypoxic conditions can comprise up to 7%, up to 6%, up to 5%, up to 4%, up to 3%, up to 2%, or up to 1% oxygen. In some embodiments, hypoxic conditions comprise about 1% oxygen up to about 7% oxygen. For example, hypoxic conditions can comprise about 1% oxygen up to about 7% oxygen; about 2% oxygen up to about 7% oxygen; about 3% oxygen up to about 7% oxygen; about 4% oxygen up to about 7% oxygen; about 5% oxygen up to about 7% oxygen; or about 6% oxygen up to about 7% oxygen. As another example, hypoxic conditions can comprise 1% oxygen up to 7% oxygen; 2% oxygen up to 7% oxygen; 3% oxygen up to 7% oxygen; 4% oxygen up to 7% oxygen; 5% oxygen up to 7% oxygen; or 6% oxygen up to 7% oxygen. As another example, hypoxic conditions can comprise about 1% oxygen up to about 7% oxygen; about 1% oxygen up to about 6% oxygen; about 1% oxygen up to about 5% oxygen; about 1% oxygen up to about 4% oxygen; about 1% oxygen up to about 3% oxygen; or about 1% oxygen up to about 2% oxygen. As another example, hypoxic conditions can comprise 1% oxygen up to 7% oxygen; 1% oxygen up to 6% oxygen; 1% oxygen up to 5% oxygen; 1% oxygen up to 4% oxygen; 1% oxygen up to 3% oxygen; or 1% oxygen up to 2% oxygen. As another example, hypoxic conditions can comprise about 1% oxygen up to about 7% oxygen; about 2% oxygen up to about 6% oxygen; or about 3% oxygen up to about 5% oxygen. As another example, hypoxic conditions can comprise 1% oxygen up to 7% oxygen; 2% oxygen up to 6% oxygen; or 3% oxygen up to 5% oxygen. In some embodiments, hypoxic conditions can comprise no more than about 2% oxygen. For example, hypoxic conditions can comprise no more than 2% oxygen.

In various embodiments, oxygen level in cell culture can be monitored according to methods well known in the art (e.g., Jung et al. (1992) Biotechnology Techniques 6: 405-408; Fleischaker and Sinskey (1981) Applied Microbiology and Biotechnology 12: 193-197).

In various aspects, pre-growing a culture of MSCs under hypoxic conditions can result in a cell culture comprising an enhanced percentage of Oil Red O-staining-cells compared to a control culture pre-in normoxic conditions. In other aspects, pre-growing a culture of MSCs under hypoxic conditions can result in a cell culture comprising an enhanced percentage of Alcian Blue-staining-cells compared to a control culture pre-grown in normoxic conditions. In yet other aspects, pre-growing a culture of MSCs under hypoxic conditions can result in a cell culture comprising an enhanced percentage of Von Kossa-staining-cells compared to a control culture not pre-grown in hypoxic conditions.

In various aspects, pre-culture of MSCs under hypoxic conditions can occur for a period of time sufficient to increase numbers of MSCs, percentage of MSCs, increase expression of MSC differentiation markers, enhance percentage of Oil Red O-staining-cells, enhance percentage of Alcian Blue-staining-cells, and/or enhance percentage of Von Kossa-staining-cells. In some embodiments, MSCs can be cultured under hypoxic conditions up to about 100 days, or longer. For example, MSCs can be cultured under hypoxic conditions up to about 21 days. As another example, MSCs can be cultured under hypoxic conditions up to about 14 days. As another example, MSCs can be cultured under hypoxic conditions up to about 13 days, about 12 days, about 11 days, about 10 days, about 9 days, about 8 days, about 7 days, about 6 days, about 5 days, about 4 days, about 3 days, about 2 days, or about 1 day. As another example, MSCs can be cultured under hypoxic conditions from about 1 day up to about 14 days; about 2 days up to about 14 days; about 3 days up to about 14 days; about 4 days up to about 14 days; about 5 days up to about 14 days; about 6 days up to about 14 days; about 7 days up to about 14 days; about 8 days up to about 14 days; about 9 days up to about 14 days; about 10 days up to about 14 days; about 11 days up to about 14 days; about 12 days up to about 14 days; or about 13 days up to about 14 days. As another example, MSCs can be cultured under hypoxic conditions from about 6 days up to 14 days; about 7 days up to 13 days; about 8 days up to 12 days; or about 9 days up to 11 days.

Culturing of mesenchymal stem cells in accordance with the present teachings can include maintenance of suitable carbon dioxide levels in the atmosphere of cell cultures. Determination of suitable carbon dioxide levels can be determined by methods known to those of skill in the art. In some embodiments, a cell culture atmosphere can comprise about 5% CO₂.

Hypoxic culture can be accomplished with any of a variety of culture chambers known in the art, such as, for example, ProOxC (BioSpherix, Lacona, N.Y.); Hypoxic Glove Box (Coy Laboratory Products, Inc., Grass Lake, Mich.); HypOxystation (HypOxygen, Frederick Md.); or Hypoxia Chamber (StemCell Technologies, Inc., Vancouver, BC).

Normoxic Conditions

Normoxic conditions generally include oxygen levels normative for culturing of cells, such as MSCs. Except as otherwise provided herein, culture of cells under normoxic conditions can utilize methods, apparatuses and components known to persons of skill in the art (e.g., Vunjak-Novakovic and Freshney (2006) Culture of Cells for Tissue Engineering, Wiley-Liss, ISBN-10 0471629359; Challen and Little (2006) Stem Cells 24(1), 3-12; Lanza et al., eds. (2004) Handbook of Stem Cells, Academic Press, ISBN 0124366430; Lanza et al., eds. (2005) Essentials of Stem Cell Biology, Academic Press, ISBN 0120884429; Saltzman (2004) Tissue Engineering: Engineering Principles for the Design of Replacement Organs and Tissues, Oxford ISBN 019514130X; Minuth et al. (2005) Tissue Engineering: From Cell Biology to Artificial Organs, John Wiley & Sons, ISBN 3527311866). Such methods can be utilized directly or adapted for use as normoxic culture conditions.

In various aspects of the present teachings, a hypoxic atmosphere in which MSCs are grown or maintained can be replaced with a normoxic atmosphere. In some embodiments, cells can grow under hypoxic conditions and express markers indicative of stem cells, and can differentiate under normoxic conditions, i.e., express markers indicative of a differentiated cell type. Duration of maintaining a culture under hypoxic conditions can be determined by routine experimentation by a person of skill in the art. Similarly, duration of maintaining a culture under normoxic conditions following hypoxic culture can be determined by routine experimentation by a person of skill in the art.

Medium

MSC culture media formulations are well known in the art (see e.g. see e.g., Vunjak-Novakovic and Freshney (2006) Culture of Cells for Tissue Engineering, Wiley-Liss, ISBN-10 0471629359; Challen and Little (2006) Stem Cells 24(1), 3-12; Lanza et al., eds. (2004) Handbook of Stem Cells, Academic Press, ISBN 0124366430; Lanza et al., eds. (2005) Essentials of Stem Cell Biology, Academic Press, ISBN 0120884429; Saltzman (2004) Tissue Engineering: Engineering Principles for the Design of Replacement Organs and Tissues, Oxford ISBN 019514130X; Minuth et al. (2005) Tissue Engineering: From Cell Biology to Artificial Organs, John Wiley & Sons, ISBN 3527311866). Except as otherwise noted herein, therefore, an MSC medium can be in accordance with practices known in the art.

Proliferation Rate of a Cell Culture Ex Vivo

In some aspects, the present teachings include methods of increasing proliferation rate of mesenchymal stem cells culture ex vivo. In these aspects, the methods can comprise providing mesenchymal stem cells in an ex vivo culture, and growing the cells under hypoxic conditions, wherein the proliferation rate of the cell culture is greater than that of a control cell culture grown under normoxic conditions. The mesenchymal stem cells can be, for example, AT-MSCs, BM-MSCs, P-MSCs or TT-MSCs.

Maintaining Mesenchymal Stem Cells in an Undifferentiated State

Some aspects of the present teachings include methods of maintaining mesenchymal stem cells in an undifferentiated state. In these aspects, the methods can comprise providing mesenchymal stem cells in an ex vivo culture, and growing the cells under hypoxic conditions. The mesenchymal stem cells can be, for example, AT-MSCs, BM-MSCs, P-MSCs or TT-MSCs.

Method of Repairing or Augmenting a Tissue or Organ in a Subject

Additional aspects of the present teachings include therapeutic treatments of a subject. In various embodiments, such treatments can comprise providing a cell culture comprising mesenchymal stem cells such as AT-MSCs, BM-MSCs, P-MSCs or TT-MSCs that has been exposed to hypoxic conditions, and transplanting the cells to a subject, such as a human subject in need of treatment or desirous of treatment.

A determination of a need for treatment can be assessed by a history and physical exam consistent with the tissue or organ defect at issue. Subjects with an identified need of therapy include, without limitation, those with a diagnosed tissue or organ defect. The subject can be a mammal or an avian, such as, without limitation, a human, a horse, a cow, a companion animal such as a dog or a cat, an agricultural animal such as a sheep, a pig, or a chicken, or a laboratory animal such as a mouse, a guinea pig or a rat.

For example, and without limitation, a subject can have a disease, disorder, or condition, for which the present methods provide a cell population, a tissue or an organ that can ameliorate or stabilize the disease, disorder, or condition. For example, the subject can have a disease, disorder, or condition that results in the loss, atrophy, dysfunction, and/or death of cells. Exemplary conditions that can be treated using cells cultured under the hypoxic conditions described herein include neural, glial, or muscle degenerative disorders, such as muscular atrophy or dystrophy, multiple sclerosis, heart disease such as congenital heart failure, hepatitis or cirrhosis of the liver, an autoimmune disorder, diabetes, cancer, a congenital defect that results in the absence of a tissue or organ, or a disease, disorder, or conditions that requires the removal and/or replacement of a tissue or organ, an ischemic disease such as angina pectoris, myocardial infarction or ischemic limb, or accidental tissue defect or damage such as a fracture or wound. In a further example, a subject in need can have an increased risk of developing a disease, disorder, or condition that can be delayed or prevented by the method. In some embodiments, a treatment can be reparative or cosmetic, such as, for example, breast augmentation can involve transplantation to a recipient of AT-mesenchymal stem cells or BM-mesenchymal stem cells grown and/or differentiated ex vivo under hypoxic conditions, and/or can be further subjected to normoxic conditions ex vivo under conditions as set forth herein.

In various configurations, a target tissue or organ of a recipient of MSC's grown under conditions as described herein can be from any organ or tissue such as, without limitation, bladder, brain, nervous tissue, glia, esophagus, fallopian tube, heart, pancreas, intestines, gall bladder, kidney, liver, lung, ovaries, prostate, spinal cord, spleen, stomach, testes, thymus, thyroid, trachea, urogenital tract, ureter, urethra, uterus, breast, skeletal muscle, skin, adipose, bone, and cartilage. MSCs such as AT-MSCs that can be transplanted to a recipient subject can be from a cell culture comprising cells originally obtained from the subject. These cells can be grown ex vivo under hypoxic conditions and/or differentiated ex vivo under hypoxic conditions. In some configurations, the cells grown under hypoxic conditions can be subjected to normoxic conditions ex vivo. In various aspects, a donor source of MSCs such as AT-MSCs that are subjected to hypoxic conditions ex vivo according to the disclosed methods, and are transplanted to a recipient can be MSCs from the same individual as the recipient (in an autologous transplantation), or can be MSCs from one or more individuals of the same species as the recipient, or can be MSCs from one or more individuals of different species as the recipient.

Various diseases or conditions that can be treated and/or ameliorated by transplanting AT-MSCs subjected to hypoxic conditions and/or differentiated ex vivo under hypoxic conditions, and/or subjected to normoxic conditions ex vivo in accordance with the present teachings include, without limitation, gastric ulcer, heart regeneration (Hu X et al., 2008), wounds (Yoshikawa T et al., 2008; Wu Y et al., 2007), lacerations, tissue repair including adipose tissue repair (Stosich M S & Mao J J, 2007), vocal fold repair (Lo Cicero, V. et al., Cell Prolif. 41: 460-473, 2008), breast augmentation, or beta cell deficiency. In various aspects, the time duration following isolation from a donor source for culture of MSCs such as AT-MSCs in hypoxic conditions, culture in normoxic conditions, exposure to growth factors and/or other differentiation factors can vary according to particular application. Determination of optimal culture times ex vivo is within the skill of the art.

In various aspects, a composition for delivery of differentiated cells described herein can further comprise a pharmaceutical carrier, preferably an aqueous carrier. A variety of aqueous carriers can be used, e.g., buffered saline and the like. The compositions can further contain pharmaceutically acceptable auxiliary substances as required to adjust culture conditions. For example, an aqueous carrier can include buffers for adjusting pH, toxicity adjusting agents, salts such as sodium acetate, sodium chloride, potassium chloride, calcium chloride, and/or sodium lactate, proteins such as albumin, anticoagulants such as CPD (citrate, phosphate, and dextrose), dextran, DMSO, and combinations thereof.

In some aspects, transplantation of cells or tissue or organ constructs of the present teachings can be accomplished according to methods well known to skilled artisans. Therapeutic differentiated or partially differentiated mesenchymal stem cells can be administered into a subject using standard methods (see e.g., Orlic et al. (2001) Nature 410(6829) 701-705). Implantation of a cell-containing composition is within the skill of a person of skill in the art. For example, differentiated or partially differentiated mesenchymal stem cells such as AT-MSCs, or compositions comprising differentiated or partially differentiated MSCs, can be introduced to a subject via direct injection such as intravenous transfusion, catheter-based delivery, or surgical implantation.

In some aspects, differentiated or partially differentiated mesenchymal stem cells can be transplanted along with a carrier material, such as collagen or fibrin glue or other scaffold materials. Such materials can improve cell retention and integration after implantation. Such materials and methods for employing them are known in the art (see e.g., Saltzman (2004) Tissue Engineering: Engineering Principles for the Design of Replacement Organs and Tissues, Oxford ISBN 019514130X; Vunjak-Novakovic and Freshney, eds. (2006) Culture of Cells for Tissue Engineering, Wiley-Liss, ISBN 0471629359; Minuth et al. (2005) Tissue Engineering: From Cell Biology to Artificial Organs, John Wiley & Sons, ISBN 3527311866).

In some aspects, an amount of differentiated or partially differentiated mesenchymal stem cells introduced into the heart tissue of the subject can be an amount sufficient to improve cardiac function, increase cardiomyocyte formation, and/or increase mitotic index of cardiomyocytes. For example, an effective amount is sufficient to increase cardiomyocyte formation, increase cardiomyocyte proliferation, increase cardiomyocyte cell cycle activation, increased mitotic index of cardiomyocytes, increase myofilament density, increase borderzone wall thickness, or a combination thereof. Improving or enhancing cardiac function generally refers to improving, enhancing, augmenting, facilitating or increasing the performance, operation, or function of the heart and/or circulatory system of a subject. In various configurations, an improvement in cardiac function can be readily assessed and determined by the skilled artisan, based on known procedures, including but not necessarily limited to, measuring volumetric ejection fraction using MRI.

In various aspects, the methods described herein can be practiced in conjunction with existing therapies to effectively treat or prevent disease. The methods or compositions described herein can include concurrent or sequential treatment with one or more of enzymes, ions, growth factors, non-biologic agents, and biologic agents, such as thrombin and calcium, or combinations thereof.

In some embodiments, differentiated cells are selected from the group consisting of adipocyte lineage cells, osteocytic lineage cells, chondrogenic lineage cells and a combination thereof. In some embodiments, the tissue or organ in the subject is selected from the group consisting of bone, skin, breast and a combination thereof. In some embodiments, the tissue or organ can be, for example, breast, cheek, chin, lips, vocal folds, heart, or stomach.

In some embodiments, the terms “a” and “an” and “the” and similar references used in the context of describing a particular embodiments (especially in the context of certain of the following claims) can be construed to cover both the singular and the plural. All methods described herein can be performed in any suitable order unless otherwise indicated herein or otherwise clearly contradicted by context.

Methods and compositions described herein utilize laboratory techniques well known to skilled artisans. Such techniques can be found in laboratory manuals such as Sambrook, J., et al., Molecular Cloning: A Laboratory Manual, 3rd ed. Cold Spring Harbor Laboratory Press, Cold Spring Harbor, N.Y., 2001; Spector, D. L. et al., Cells: A Laboratory Manual, Cold Spring Harbor Laboratory Press; Cold Spring Harbor, N.Y., 1998; Harlow, E., Using Antibodies: A Laboratory Manual, Cold Spring Harbor Laboratory Press, Cold Spring Harbor, N.Y., 1999; Methods of administration of pharmaceuticals and dosage regimes, can be determined according to standard principles of pharmacology well known skilled artisans, using methods provided by standard reference texts such as Remington: the Science and Practice of Pharmacy (Alfonso R. Gennaro ed. 19th ed. 1995); Hardman, J. G., et al:, Goodman & Gilman's The Pharmacological Basis of Therapeutics, Ninth Edition, McGraw-Hill, 1996; and Rowe, R. C., et al., Handbook of Pharmaceutical Excipients, Fourth Edition, Pharmaceutical Press, 2003. These publications are incorporated herein by reference, each in its entirety.

### EXAMPLES

The following non-limiting examples are provided to further illustrate the present teachings and are not intended to limit the scope of any claim. Unless specifically presented in the past tense, an example can be a prophetic or an actual example.

In some examples, results are presented as mean±standard error (SE). Statistical significance between two measurements was evaluated by Student's t test. A probability value of p<0.05 was considered significant.

### Example 1

### MSC Isolation and Culture

Mesenchymal stem cells (MSC) were isolated from bone marrow (BM), adipose tissue (AT), pancreas (P) and testis tissue (TT) of 8-12 week-old non obese diabetic (NOD) male mice. At this stage, mice had not developed diabetes as assessed by the evaluation of their glucose levels using a hand-held glucometer (Accu-Chek Tests, Roche Diagnostics GmbH, Mannheim, Germany) (which, in non diabetic mice are <11.5 mmol/L). The NOD mice, the breeding and the stock were housed in individually ventilated cages with exhaust system (Sealsafe IVC) and on the relevant safety standards. Mice were kept in specific pathogen-free conditions, in a controlled temperature (maintained at 21° C.), relative humidity at 50% and were given autoclaved food and water ad libitum. The NOD mice were sacrificed by cervical dislocation according to UK Home Office regulations.

BM cells were collected by flushing femurs, tibias and iliac crests with 5 ml PBS supplemented with 2% fetal bovine serum (FBS; Gibco, Paisley, UK). AT cells were obtained from the epiploon that was excised, cut into small pieces, digested for 2 hrs at 37° C. with sharing every 15 mins, with the digestion medium (0.5 gr/ml) consisting of DMEM (Gibco, Invitrogen Corporation, Carlsbad, Calif.) with 1 mg/ml of collagenase A (Roche Diagnostics GmbH, Mannheim, Germany) and cells were centrifuged and filtered through a 40 μm nylon filter (Becton Dickinson Labware, Franklin Lakes, N.J., USA). Pancreas-derived MSC (P-MSC) and testis tissue-derived MSC (TT-MSC) were isolated as AT-MSC. Cells were plated at a density of 1×10⁵ cells/cm² and cultured in Complete Medium: Murine Mesenchymal Medium with 20% Murine Mesenchymal Supplements (Stem Cell Technologies, Vancouver, Canada) further supplemented with 100 IU/ml penicillin and 100 μg/ml streptomycin (Gibco, Paisley, UK). Cells were incubated at 37° C. in a humidified 5% CO, atmosphere in 21% oxygen (normoxia). Hypoxic conditions were created using an Invivo2 1000 hypoxia workstation (Ruskinn Technology Ltd., Pencoed, Wales) according to the manufacturer's instructions. The workstation's atmosphere was continually monitored for CO₂ and O₂ concentrations and adjusted by adding a mixture of 3 gases (compressed medical air, medical N₂ and medical CO₂). A final and maintained concentration of 2% O₂, 5% CO₂ was achieved before placing the cultures in the workstation. The workstation was kept at 37.5° C. with humidity set above 90%.

In some experiments, AT cells were obtained from pooled omental fat (epiploon) of five 8-12 week NOD mice. The omental fat (epiploon) of these mice were cut into small pieces, digested for 2 hrs at 37° C. with shaking every 15 mins, with 1 mg/ml Accutase (Chemicon, Millipore). This cell detachment solution of proteolytic and collagenolytic enzymes was used for gentle tissue digestion. Cells were centrifuged and filtered through a 40 μm nylon filter (Becton Dickinson Labware, Franklin Lakes, N.J., USA). P- and T-MSCs were isolated as AT-MSC.

Non-adherent cells were eliminated by a half medium change at day 1-3, washed with PBS then cultured with fresh Complete Medium. Half of the volume of medium was replaced twice a week. The whole adherent fraction was detached by trypsinization at 80% confluency using Accutase (Chemicon Europe, Hampshire, UK). In some experiments, non-adherent cells were eliminated, in normoxic as well as in hypoxic cultured cells, by a complete medium change at day 1 and a wash with PBS of the adherent cells remaining in the cultures. Then, cells were cultured with fresh Complete Medium and a half volume of medium was replaced twice a week. The whole adherent fraction was detached by trypsinization at 80% confluence (after 4-5 days) using Accutase (Chemicon Europe, Hampshire, UK) and re-plated.

### Example 2

### Flow Cytometry Analysis

In some experiments, FACS analysis was performed at day 5 (after 10 days of exposure to normoxia and hypoxia). The phenotype of cultured BM-MSCs, AT-MSCs, P-MSCs and T-MSCs was analyzed by Fluorescence Activated Cell Sorter (FACS) analysis using a BD LSR II analyzer or a BD FACSAria analyzer fitted with DIVA software.

The following rat anti-mouse IgG monoclonal antibodies were used: Fluorescent isothiocyanate (FITC)-conjugated and phycoerythrin-cyanin7 (PECy7)-conjugated Sca-1; FITC-conjugated CD44. Negative selection was performed with phycoerythrin (PE)-conjugated CD45, CD11b, TER119 and CD31 rat anti-mouse IgG (BD Biosciences Pharmingen, Palo Alto, Calif., USA). FACS analysis was performed on hematopoietic and endothelial lineage-negative cells (Anjos-Afonso et al., J Cell Sci 117, 5655-5664, 2004) which were identified following incubation with phycoerythrin (PE)-conjugated CD45, CD11b, TER119 and CD31 rat anti-mouse IgG (BD Biosciences Pharmingen, Palo Alto, Calif., USA). As controls, cells were stained with FITC, PECy7, PE-labeled isotype rat anti-mouse IgG. The compensation was performed using single colour controls. Samples were analyzed to compare the negative selection antibodies against Sca-1-PE-Cy7 or CD44-FITC. CD44⁺/Negative Selection were then gated to show percent double-positive for CD44 and Sca-1.

### Example 3

### In Vitro Adipogenic, Osteogenic and Chondrogenic Differentiation

For adipogenic differentiation, both BM- and AT-MSC were cultured in Complete Medium with 0.5 μM hydrocortisone, 0.2 μM isobutyl methyl xanthine, 100 μM indomethacin and 5 μg/ml insulin (Nagai et al., PLoS ONE 2: 543 e1272, 2007). The culture medium was changed three times per week for up to 3 weeks. Then cells were fixed with 4% PFA in PBS for 20 minutes at room temperature, incubated in 60% iso-propyl-alcohol (IPA) and stained with 1% Oil Red O (Raymond Lamb, Eastbourne, UK) in IPA for 15 minutes, and further incubated in IPA to remove background staining. Nuclei were stained with half-strength Harris' hematoxylin for 30 seconds, then mounted in Glycergel. The positive fat vacuoles appeared as red stained droplets.

Chondrogenesis was assessed by culturing cells for up to 3 weeks in Complete Medium containing 1 ng/ml bFGF and 5 ng/ml TGF-β1. Chondrocytes were stained with 1% alcian blue (BDH, Poole UK) in 3% acetic acid, pH 2.5 for 5 minutes, with a 1 minute neutral red nuclear counterstain, which revealed sulphated proteoglycan production by MSCs (Mouiseddine et al., Br J Radiol. 80 Spec No 1: S49-55, 2007).

For osteogenic differentiation, cells were grown for up to 3 weeks in Complete Medium supplemented with 10 nM dexamethasone, 0.2 mM vitamin C phosphate and 10 mM Na□-β-glycerophosphate. Von Kossa staining for calcium salts was used to detect osteocytes as described (Bancroft, C., and Gamble, M., eds. Theory and Practice of Histological Techniques. 5th ed Edinburg, U.K.: Churchill Livingstone, 2002 p 293).

### Example 4

### RNA Isolation, Array Analysis and qPCR

Total RNA was extracted using RNeasy Mini kit (Qiagen AG, Hilden, Germany). Taqman RNA to Ct 2 step kit (Applied Biosystems, Warrington, UK) was used for reverse transcription of total RNA (1 μg) into complementary DNA and quantitative PCR according to the manufacturer's instructions. The following gene specific assays (Applied Biosystems) were used: Nanog (Mm02019550_sl); Sox2 (Mm00488369_sl); Oct4 (Mm00658129_gh); Pparγ (Mm00440945_ml); Lpl (Mm00434764_ml); Fabp4 (Mm00445880_ml). Expression levels were normalized against Gapdh using Mouse Gapdh TaqMan as an endogenous control (Applied Biosystems) and as a reference control for quantitative PCR gene-expression analysis. To assess the linearity and sensitivity of the assay, a standard curve was generated using serial dilutions of Stratagene QPCR Mouse Reference Total RNA (Stratagene, Calif., USA). qPCR measurements were performed in triplicate. All quantitative PCR were carried out using a 7500 Real-Time instrument (Applied Biosystems). The amplified transcripts were quantified using the comparative CT method with the formula for relative fold change=2^{-ΔΔCT}.

### Example 5

### Proliferation Studies

In some experiments, normoxic and hypoxic AT-MSCs were isolated from a pool of 8 weeks old Balb/c male mice (n=5), cultured in normoxic and hypoxic conditions for 5 days, then detached and plated in 12.5 cm² flasks for the indicated time points (3000 cells/cm²). Cells were used at passage P1.

### Example 6

### Apoptosis Studies

In some experiments, normoxic and hypoxic cultured AT-MSCs were trypsinized, resuspended in 200 μl of calcium rich annexin V buffer (BD Biosciences, Oxford, UK) and incubated 15 minutes at RT with 15 μl of annexin V-AlexaFluor-647 (Invitrogen, Paisley, UK). Propidium iodide (P1) (5 μg/ml) was added and samples were analysed on a Becton Dickinson LSRII cytometer, using the 660/20 nm channel from the red laser for annexin V-AlexaFluor-647 detection and the 576/26 nm channel from the argon laser was used to detect P1 (10.000 events were collected). No compensation controls were required as P1 and AlexaFluor-647 did not spectrally overlap. Quadrant gating was used to detect live cells (annexin V neg/P1neg), apoptotic cells (annexin V pos/P1neg), and dead cells (annexin V neg/P1pos) and (annexin V pos/P1pos).

For cell cycle distribution analysis, annexin V labelled cell were fixed in 70% ice-cold ethanol, spin-washed in PBS and incubated with 100 μg/ml RNAse (Sigma) at 37° C. for 15 minutes and resuspended in 50 μg/ml P1 in PBS. Then, samples were analysed (10.000 events collected) on a Becton Dickinson LSRII cytometer using the 610/10 nm channel from the argon laser to detect P1 in a linear manner with the width parameter used to exclude doublets of cells. Histogram analysis of the P1 signal allowed the determination of the percentage of cells that have lost DNA due to DNA fragmentation. The result was a population of cells with a reduced DNA content and the cells were stained with a DNA intercalating dye, P1. A DNA profile representing cells in G1, S-phase and G2M was observed with apoptotic cells being represented by a Sub G1 population seen to the left of the G1 peak.

### Example 7

### FACS Analysis of Cell Surface Antigen Expression

Except as otherwise noted, methods in this and following examples are in accordance with Examples 1-6.

In this example, MSCs were isolated from the epiploon of 8-12 week old NOD mice. AT was excised, collagenase digested and filtered. The cells were grown under atmospheric (21%) or hypoxic (2%) levels. Isolated cells were phenotyped by flow cytometry (FACS) for surface antigen expression mesenchymal stem cell markers CD44 and Sca1 (as evidence of MSCs) (see, e.g., Sung, J. H., et al., Transplant Proc. 40: 2649-2654, 2008).

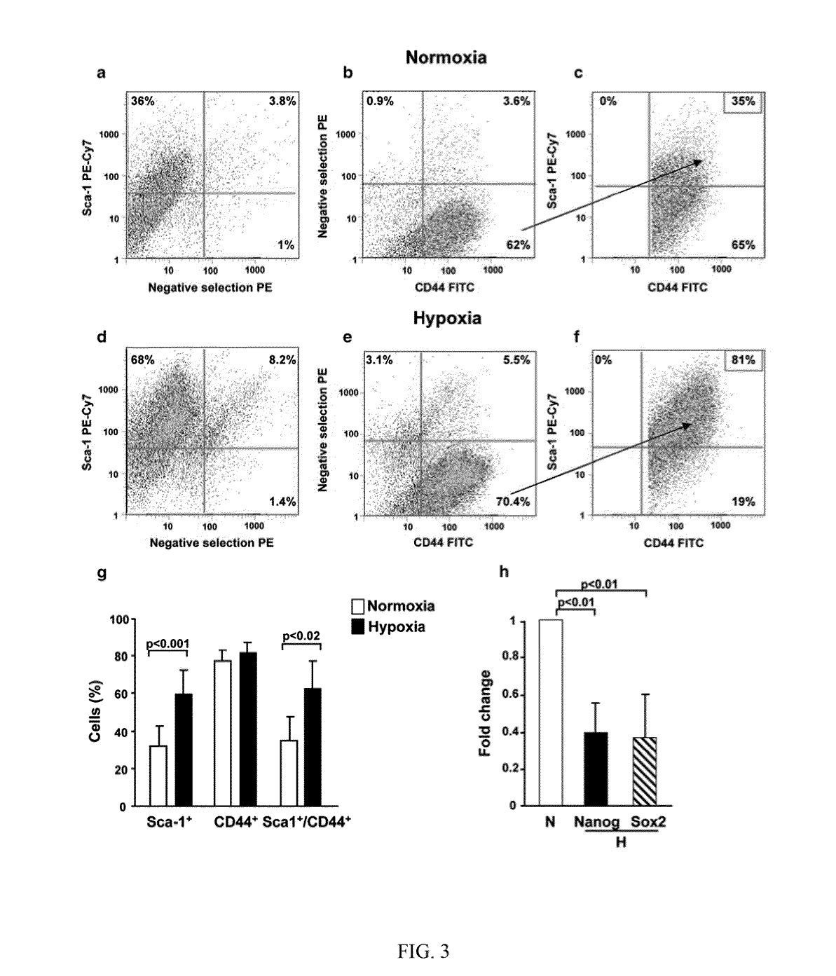

FACS analysis of MSCs showed that while CD44 was highly expressed by cells grown under either hypoxic and normoxic conditions, Sca-1 strongly increased under hypoxia. After 10 days of hypoxic culture, 81% of MSCs co-expressed Sca-1 and CD44, but only 35% of MSCs were double-positive in normoxia (FIG. 3).

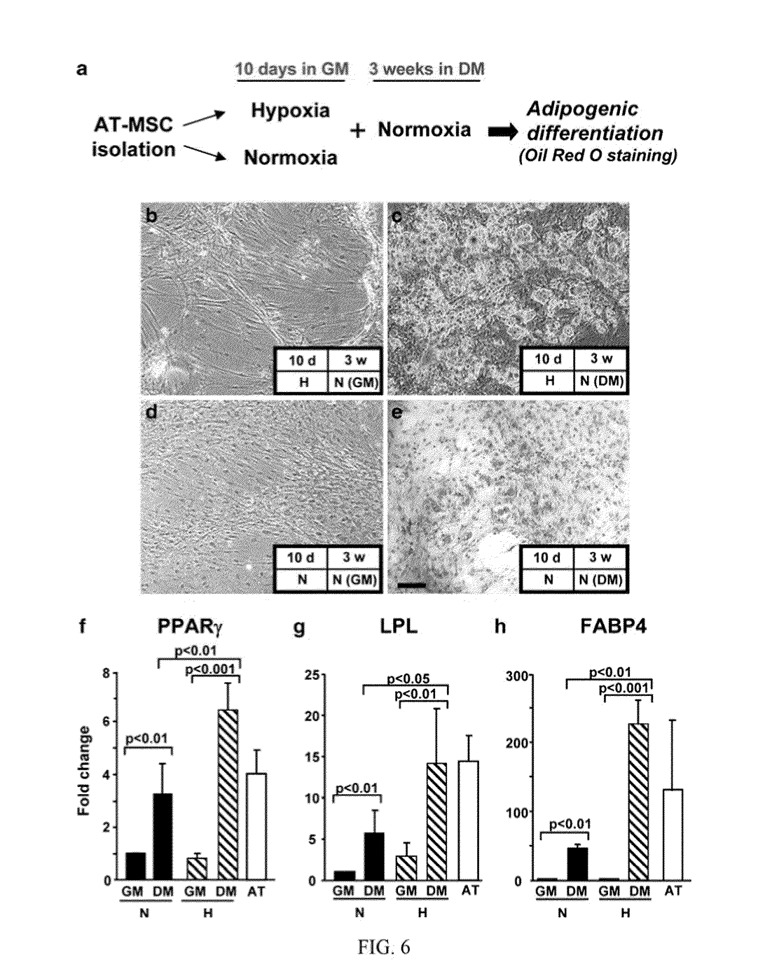

We also compared the capacity of normoxic and hypoxic grown cells to differentiate into fat in normoxia. Our data show that previously hypoxic cultured MSCs displayed higher adipogenic differentiation compared to normoxic-cultured MSCs as confirmed by increased expression of adipogenic differentiation genes PPARγ, LPL and FABP4, as determined using Real-Time PCR analysis (FIGS. 6f-h; see Example 13).

These results demonstrate that prior exposure to hypoxic culture conditions maintains MSCs such as AT-MSCs in a more undifferentiated state, increases substantially the proliferation rate of cells cultured, enhances their purity and consequently minimizes the time required for their ex vivo expansion and also enhances their differentiation potential. Culturing MSCs under hypoxia therefore represents an effective strategy to increase the MSC pool. MSCs cultured under these conditions therefore provide a source of cells for tissue engineering. Therapies using these cells can be performed in many medical contexts, such as after oncological resections and complex traumas or augmentative surgery of the breast, cheek, chin or lips.

### Example 8

### Demonstration that Isolated Cells are True MSC Populations

Except as otherwise noted, methods are according to Examples 1-7.

MSC were isolated from both BM and AT of 8-12 week old NOD mice. Briefly, BM cells were collected by flushing femurs, tibias and iliac crests while AT cells were obtained from the epipolon which was excised, cut into small pieces, collagenase digested and filtered. The cells were grown under atmospheric (21%) or low oxygen levels (2%).

To confirm that isolated cells were true MSC populations, in vitro differentiation into adipocytic, osteocytic and chondrocytic phenotypes was carried out. The BM-MSCs and AT-MSCs were both capable of trilineage differentiation when grown in specific media.

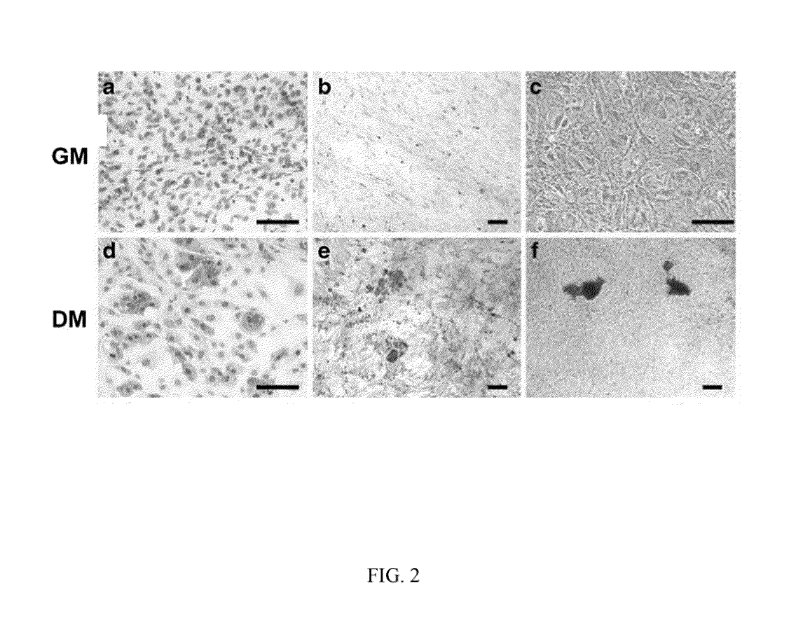

In these experiments, to confirm that in vitro cultured cells maintained MSC potential, we investigated their ability to differentiate along the osteogenic, adipogenic and chondrogenic lineages. FIG. 2 illustrates differentiation potential of normoxic cultured AT-MSC. AT-MSC differentiation toward adipogenic, chondrogenic, osteogenic lineages in culture. Representative images of AT-MSC cultured under normoxic conditions for 10 days in growth control medium (GM, upper panels) (FIGS. 2a-c) and later cultured for 3 weeks in the specific differentiation cocktail media (DM, lower panels) (FIGS. 2d-f). Adipogenic cells were identified by Oil Red O staining of intracellular lipid droplets (FIG. 2d). Alcian blue staining revealed sulphated proteoglycan production by MSCs showing chondrogenic differentiation (FIG. 2e). Von Kossa staining for calcium salts was used to detect osteocytes (FIG. 2f). Bars 100 μm. In these experiments, AT-MSCs were grown in normoxia for 10 days and after that, cultured for 3 weeks in either growth media (FIGS. 2a-c) or specific differentiation media FIGS. 2d-f). These studies showed that AT-MSC are capable of giving rise to adipocytes, as visualized by intracellular lipid droplets using Oil Red O staining (FIG. 2d), chondrocytes, had sulphated proteoglycan production confirmed by alcian blue staining (FIG. 2e) and osteogenic cells with calcium salt deposition were identified by von Kossa staining (FIG. 2f).

### Example 9

### Effects of Hypoxia on Bone Marrow MSCs

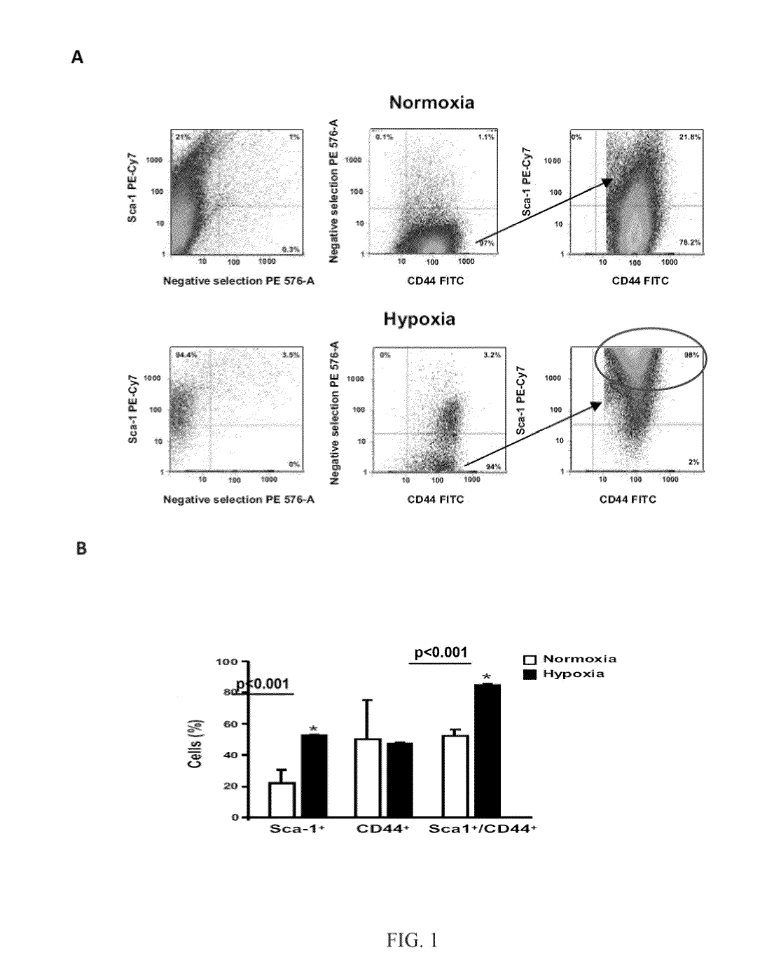

This example shows the effects of hypoxia on bone marrow MSCs (BM-MSCs). FIG. 1 illustrates fluorescence-activated cell sorting (FACS) analysis of bone marrow MSCs at 86-92 days (P8) in culture in normoxia and hypoxia conditions. Representative dot plots (A) and histograms with percentage of Sca1⁺, CD44⁺ cells and Sca1⁺/CD44⁺ cells detected (B). Sca1/CD44 double positive cells increase in BM cultured under hypoxia conditions. (A) Results from FACS analysis of bone marrow MSCs at 86-92 days (P8) in culture in normoxia and hypoxia. Representative FACS results are shown. (B) Histogram of the results from (A), in which the percentage of Sca1⁺, CD44⁺ and Sca1⁺/CD44⁺ cells are quantified. The data demonstrate that when BM-MSCs are grown in hypoxic conditions, more cells become or remain Sca1⁺/CD44⁺ than when BM-MSCs are grown in normoxic conditions. In these experiments, isolated cells were phenotyped by flow cytometry (FACS) for surface antigen expression of CD44 and Sca-1 (as evidence of murine MSCs). FACS analysis of cultured BM-MSCs and AT-MSCs showed that when grown in hypoxic conditions, Sea-1⁺ cells were increase in both populations. After 90 days, 98% of hypoxic-grown BM-MSCs were Sca-1⁺/CD44⁺, whereas from normoxic culture only 22% were Sca-1⁺/CD44⁺ (FIG. 1).

### Example 10

### Effects of Hypoxia on Adipose Tissue-Derived MSCs

The data presented in this example demonstrate that Sca1/CD44 double positive cells increase in AT-MSCs cultured under hypoxia conditions.

To evaluate the effects of low oxygen levels, AT-MSCs were cultured in parallel both in normoxia (21% O₂) and in hypoxia (2% O₂). FIG. 3 illustrates FACS analysis of adipose tissue-derived MSCs after 10 days (P1) in culture in normoxic and hypoxic conditions. Representative dot plots (FIGS. 3a-f) and histograms are shown, with percentage of Sca1⁺, CD44⁺ cells and Sca1⁺/CD44⁺ cells detected (FIGS. 3g, h).

FACS analysis of normoxic and hypoxic cultured adipose tissue-derived MSCs at day 10 (P1) are shown in FIGS. 3a-c and FIGS. 3d-f, respectively. Representative dot plots of Sca1⁺, CD44⁺ and Sca1⁺/CD44⁺ cells are shown in FIGS. 3a,d, FIGS. 3b,e, and FIGS. 3c,f, respectively. Negative selection was performed incubating cells with phycoerythrin (PE)-conjugated CD45, CD11b, TER119 and CD31 rat anti-mouse IgG and measuring PE fluorescence at 576 nm. CD4⁴⁺ cells in the middle panels were then gated to show percent double-positive for CD44 and Sca-1.

FIG. 3g: Histogram showing results from FACS analysis (n=3) in which the percentage of Sca1⁺, CD44⁺ and Sca1⁺/CD44⁺ cells are quantified. There was a significant increase in the frequency of Sca1 positive cells when grown in hypoxia compared to normoxia (p<0.001) and likewise a significant increase in Sca1⁺/CD44⁺ (p<0.02). As shown in FIG. 3g, after only 10 days in hypoxic culture, 81% of AT-MSCs were Sca-1⁺/CD44⁺, whereas only 35% were Sca-1⁺/CD44⁺ following growth in normoxic culture.

Flow cytometric analysis performed on 10 days' AT-MSC culture showed that hypoxia significantly enhanced the frequency of Sca-1⁺ as well as Sca-1⁺/CD44⁺ cells in AT-MSC in comparison to normoxic conditions (59.5±13% vs. 32±11% and 62±15% vs. 34.5±13%) (FIGS. 3a-g).

FIG. 3h: Real-time RT-PCR showing the fold change of Nanog and Sox2 expression in AT-MSC cultured for 10 days either in hypoxic (H) and in normoxic (N) conditions (n=3). Under normoxia, the expression levels of both Nanog and Sox2 were similar: deltaCT=15.6±0.4 and 15.3±1.2, respectively. Nanog and Sox2 decreased under hypoxic conditions (p<0.01).

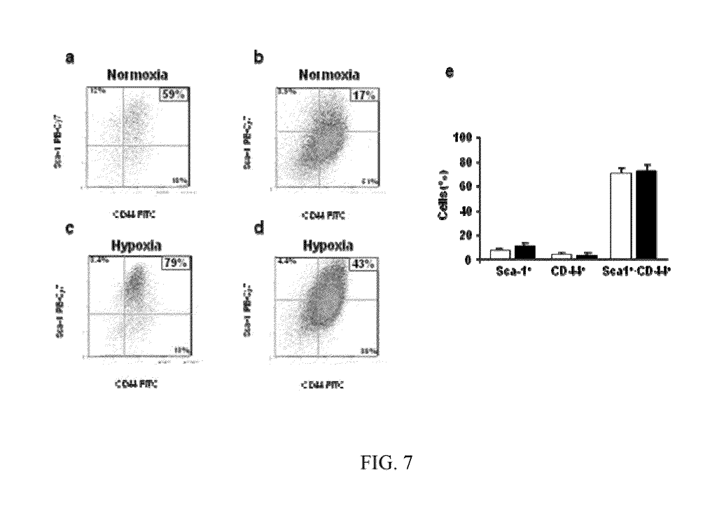

Notably, low oxygen levels enhanced the number of Sca-1⁺/CD44⁺ cells in the MSC fraction obtained from both pancreas and testis (FIGS. 7a, c and FIGS. 7b,d, respectively; see Examples 14, 15). In contrast, FACS analysis of normoxic cultured bone marrow (BM)-MSC revealed that Sca-1⁺ and CD44⁺ cells were less abundant compared to AT-MSC (7.6±2.7% vs 32±11%, p<0.05; and 3.9±3% vs 67±12%, p<0.001); these percentages did not significantly change between 20 days normoxic and hypoxic BM-MSC culture, however, after 20 days culture, 70±4% of CD44 cells also expressed Sca-1.

FIG. 7e presents a histogram showing results from FACS analysis of the percentage of Sca-1⁺, CD44⁺ cells and Sca1⁺/CD44⁺ double positive cells (n=3) in 20 days cultured BM-MSC under normoxic (open bars) and hypoxic (black bars) conditions.

The data demonstrate that, when adipose tissue-derived MSCs are grown in hypoxic conditions, more cells become or remain Sca⁺/CD4⁺ compared to adipose tissue-derived MSCs that are grown in normoxic conditions.

### Example 11

### Oxygen Levels Can Affect the Expression of Pluripotency Stem Cell Markers

We investigated whether low oxygen levels can affect the expression of pluripotency stem cell markers Nanog, Sox2 and Oct4. By Real time PCR we showed that 10 days hypoxic culture conditions can markedly inhibit Nanog and Sox2 mRNA levels in AT-MSC (FIG. 3h). In contrast, Oct-4 levels were detectable neither in normoxia nor in hypoxia. (data not shown). Previous studies have reported the opposite effect of hypoxia on Oct-4 levels in human and murine bone marrow MSC (Grayson et al., Biochem Biophys Res Commun 358: 948-953, 2007; Grayson et al., J Cell Physiol 207: 331-339, 2006; Ren et al., Biochem Biophys Res Commun 347: 12-21, 2006), however, different time of exposure to low oxygen levels may account for such discrepancy.

### Example 13

### Effects of Hypoxia on Cell Growth and Adipogenic Differentiation of AT-MSC

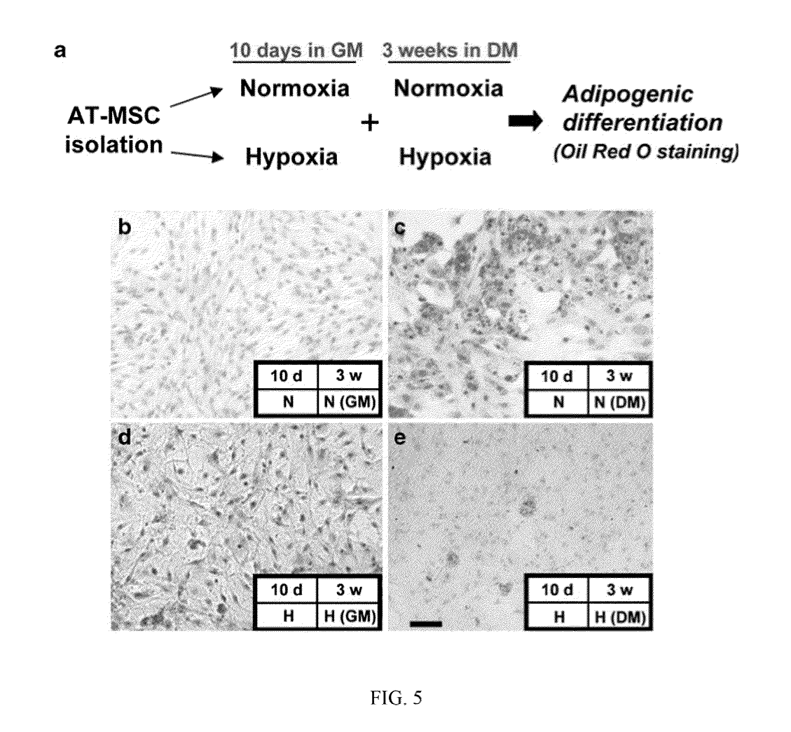

To evaluate the effects of hypoxia on MSC differentiation, the ability of normoxic and hypoxic cells to differentiate into adipogenic cells was assessed. Although hypoxia inhibited MSC differentiation into adipocytes (FIG. 5), hypoxic-cultured MSCs displayed higher adipogenic differentiation potential when transferred to normoxic conditions, compared to normoxic-cultured MSCs (FIG. 6).

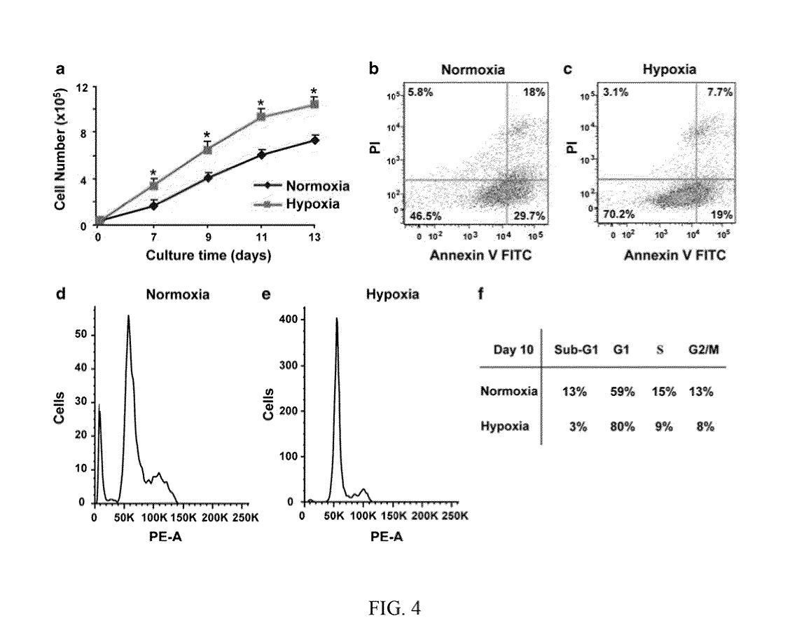

FIG. 4 illustrates Effect of hypoxia on cell growth, survival and cell cycle distribution. FIG. 4a: Growth curve of normoxic and hypoxic AT-MSC (n=4, p<0.001). Cells were detached and counted at the indicated time points. Note that at day 13 hypoxic cells were at 80-85% confluency. FIGS. 4b, c: Representative dot plots of annexin V- and P1-labeled AT-MSCs after 10 days culture either in normoxia or in hypoxia. Data representative of two independent experiments. FIGS. 4d, e: Cell cycle distribution and percent (f) of P1-labeled AT-MSCs after 10 days culture either in normoxia or in hypoxia.

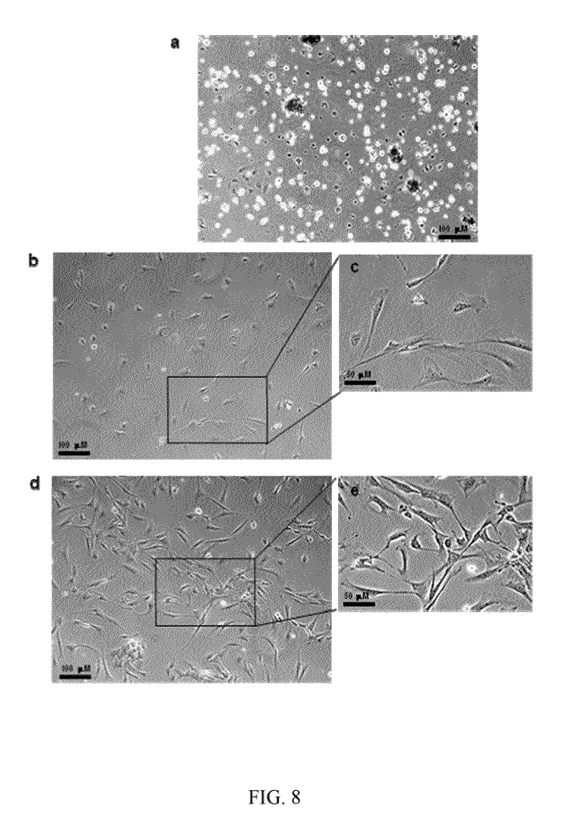

The growth curve showed that from day 7 AT-MSC number significantly increased in hypoxic cultured cells at all time points analyzed (FIG. 4a). As illustrated in FIG. 8, both hypoxic and normoxic cells exhibited a small, spindle-shaped morphology in GM-cultured murine AT-MSCs after 24 hrs in normoxia (FIGS. 8a) and 3 days either in normoxia (FIGS. 8b, c) or in hypoxia (FIGS. 8d,e), where we observed more proliferation.

Annexin V and propidium iodide (P1) staining revealed that hypoxia protected AT-MSC from death. Specifically a combination of both parameter, P1 and annexin V allowed for the discrimination between necrotic P1-positive and apoptotic P1 negative/annexin V positive cells. At day 10, the number of apoptotic cells was higher in normoxic cultured AT-MSC compared to their hypoxic counterpart (FIG. 4b, FIG. 4c). Flow cytometric analysis of cell cycle distribution was performed to further confirm the presence of apoptotic cells. Apoptosis can result in the progressive generation of particles corresponding to hypodiploid DNA content, which reflects DNA fragmentation. By flow cytometry apoptotic cells appear as a peak in ‘sub-G1’. As reported in the FIGS. 4d-f, at day ten of culture, the percentage of sub-G1 cells was lower in hypoxia compared to the normoxia counterpart. Taken together these data suggest that low oxygen levels enhanced the proliferative activity of AT-MSCs and protected them from death.

To evaluate the effect of hypoxia on adipogenic differentiation, AT-MSCs were cultured for 10 days in growth medium under either normoxic or hypoxic conditions. We then analyzed their ability to undergo adipogenic differentiation in presence of adipogenic differentiation medium (FIG. 5a). AT-MSCs were pre-cultured in growth control medium (GM) for 10 days in normoxia (FIGS. 5b, c) and hypoxia (FIGS. 5d, e). Then, cells were cultured for a period of 3 weeks either in differentiation cocktail medium (DM) (FIGS. 5c, e, right panels) or GM (FIGS. 5b,d, left panels). Oil red O staining was performed to detect adipogenic differentiation and showed lipid vacuole accumulation in normoxic-DM cultured cells (FIGS. 5c, e). Adipogenic differentiation was strongly reduced when AT-MSC were cultured in GM and seldom in adipogenic medium (DM) under hypoxia. Bar=100 μm.

As expected, Oil-Red O-positive colonies were detected in AT-MSCs pre-exposed for 10 days to normoxia and cultured for 3 weeks in adipogenic differentiation medium under normal oxygen levels (FIG. 5c). This phenomenon was strongly attenuated in AT-MSC pre-exposed for 10 days to hypoxia and then cultured for 3 weeks in adipogenic differentiation medium under hypoxic conditions (FIG. 5e), confirming previous studies that hypoxia affects the adipogenic differentiation capacity of MSCs (Fehrer et al., Aging Cell 6: 745-757, 2007; Fink et al., Stem Cells 22: 1346-1355, 2004; Lee and Kemp, Biochem. Biophys. Res. Comm. 341: 882-888, 2006).

To analyze whether the adipogenic differentiation program was temporarily or permanently inhibited by hypoxia, AT-MSCs were cultured in growth medium (GM) under normoxia or hypoxic conditions for 10 days, the cells were then transferred to normoxia and the GM was replaced with the adipogenic differentiation medium (DM) (FIGS. 6a-e). As illustrated in FIG. 6, pre-hypoxic-cultured AT-MSCs display enhanced adipogenic differentiation potential when exposed to normoxia. FIG. 6a: Experimental plan. AT-MSCs were cultured under hypoxia in growth control medium (GM) for 10 days and transferred to normoxia in the presence of GM (FIG. 6b) or the adipogenic differentiation cocktail medium (DM) (FIG. 6c) for a period of 3 weeks. As controls, AT-MSCs were cultured in normoxia and then exposed for the same time either to GM (FIG. 6d) or DM (FIG. 6e). Oil red O staining showed that lipid vacuoles accumulated to a greater extent in pre-hypoxic AT-MSCs exposed to normoxic conditions under DM (FIG. 6c) compared to pre-normoxic AT-MSCs cultured in DM (FIG. 6e). Bar=100 μm. FIGS. 6f-h: Real-time RT-PCR showing the expression of genes involved in adipogenesis Pparγ, Lpl and Fabp4, in the culture conditions described in FIGS. 6b-e. Briefly, cells pre-exposed to either normoxia (N) or hypoxia (H) for 10 days were induced to differentiate after 3 weeks of culture in adipogenic medium (DM) under normoxic conditions (n=3). As negative controls, cells were left in GM. As positive controls fresh murine adipose tissue (AT) was used for the expression of the indicated genes. Data are expressed as fold change from normoxic GM-cultured cells.

Oil Red-O staining performed after 3 weeks of differentiation showed that the cells pre-exposed to hypoxia differentiated into adipocytes to a greater extent compared to the cells pre-exposed to normoxia (FIGS. 6c, e). The expression of the adipogenic genes peroxisome proliferator activated receptor γ (Pparγ), lipoprotein lipase (Lpl) and fatty acid binding protein 4 (Fabp4) was evaluated by Real Time RT-PCR to quantify the adipogenic differentiation of normoxic and hypoxic pre-grown cells that were all transferred to normoxia. As expected, the addition of differentiation medium to pre-normoxic cultured cells resulted in increased expression of Pparγ, Lpl and Fabp4. However, when pre-hypoxic cultured AT-MSCs were exposed to differentiation medium under normoxic conditions, the expression of adipogenic genes was significantly higher compared to their normoxic counterparts (FIGS. 6f-h).

### Examples 14

### Effects of Hypoxia on Pancreatic Tissue MSCs

FIG. 7 illustrates that low oxygen levels enhances the number of Sca-1⁺/CD44⁺ cells in the MSC fractions obtained from pancreas.

In these experiments, pancreatic tissue MSCs were grown for 37 days (P1) in either normoxia (21% oxygen) or hypoxia (2% oxygen). FACS analysis of the percentages of Sca1⁺/CD44⁺ cells is shown. In cultures grown in normoxia, 59% of cells are Sca1⁺/CD44⁺, whereas in cultures grown in hypoxia, 79% of cells are Sca1⁺/CD44⁺(FIGS. 7a, c). These experiments illustrate that hypoxia enhances Sca-1⁺/CD44⁺ in pancreatic tissue-MSCs.

### Example 15

### Effects of Hypoxia on Testis Tissue MSCs

In these experiments, testis tissue MSCs were grown for 9 days (P0) in either normoxia (21% oxygen) or hypoxia (2% oxygen). FACS analysis of the percentages of Sca1⁺/CD44⁺ cells is shown (FIGS. 7b, d). In cultures grown in normoxia, 17% of cells are Sca1⁺/CD44⁺, whereas in cultures grown in hypoxia, 43% of cells are Sca1⁺/CD44⁺. These experiments illustrate that hypoxia enhances Sca-1⁺/CD44⁺ in testis tissue-MSCs.

### Example 16

### Exposure of AT-MSCs to Hypoxic Conditions Followed by Transfer to Normoxic Conditions can Enhance Adipogenic Differentiation

In these experiments, AT-MSC cultures were cultured in either normoxia or hypoxia for a pre-determined length of time. The hypoxia cultures were then transferred to normoxia, and both sets of cultures were allowed to continue growth. After a period of growth in normoxia, both sets of cells were either stained with Oil O Red, to reveal adipogenic differentiation and/or the formation of lipid vacuoles, or were analyzed by RT-PCR for expression of genes involved in adipogenesis. As illustrated in FIGS. 6b-e, Oil red O staining shows lipid vacuoles after culture in adipogenic cocktail medium in pre-normoxic AT-MSCs (left panels) and in pre-hypoxic AT-MSCs exposed to normoxic conditions for the same time. These data demonstrate that adipogenic differentiation was enhanced in cells initially cultured in hypoxic conditions and then transferred to normoxia, compared to control cells cultured in normoxia only. FIGS. 6f-h illustrates real time RT-PCR showing the expression of genes involved in adipogenesis (PPARγ, LPL and FBP4) in culture conditions described in FIG. 6a. These data demonstrate that expression of genes involved in adipogenesis, including PPARγ, LPL and FBP4 was statistically significantly higher in cells grown first in hypoxic conditions compared to cells grown only in normoxic conditions. Adipose tissue was used as controls for the expression of the indicated genes.

### Example 17

### Temporary Hypoxia Enhances Human Adipose Tissue Mesenchymal Stem Cells Adipogenic Differentiation Potential

This example illustrates that pre-culturing human adipose tissue mesenchymal stem cells under hypoxic conditions is an effective strategy to establish human multipotent cells enhanced in adipogenic differentiation potential.

In this study, the inventor evaluated whether low oxygen level (2%) affected human adipose tissue mesenchymal stem cells (hAT-MSC) proliferation and their adipogenic differentiation potential. Fat tissue was harvested from three human female donors, who had given their informed consent: No. 1, 20 years old, No. 2, 23 years old and No. 3, 55 years old. Tissue was obtained from lower abdomen curetting during suction under moderate negative pressure, using a 50-mL disposable syringe connected to a 2-holed 4.0 mm blunt cannula. The samples were generously provided by Ospedale Maggiore Policlinico, Mangiagalli e Regina Elena-Fondazione IRCCS, Milano, Italy. Lipoaspirates were washed with sterile phosphate buffered saline (PBS; Invitrogen, Carlsdad, Calif., USA) in order to remove contaminating debris and red blood cells, and then treated with 0.075% collagenase type A (Roche, Mannheim, Germany) in PBS for 30 min at 37° C. with gentle agitation. Collagenase was inactivated by an equal volume of Dulbecco's modified Eagle's medium-low glucose (DMEM-LG) (Lonza, Wokingham, UK) supplemented with 20% fetal bovine serum (FBS) (Biochrom AG, Berlin, Germany), and the suspension was centrifuged at low speed for 10 min. The stromal vascular fraction was plated overnight in normoxia in fresh complete medium: DMEM-LG/10% FBS/1% penicillin-streptomycin (Lonza, Wokingham, UK)/(Biochrom AG, Berlin, Germany)/(Gibco) after which the non-adherent fraction was removed (Lo Cicero V. et al. Cell Prolif. 2008).

Adherent human cells were cultured for 10 days in parallel in Normoxia (21% O₂) and in Hypoxia (2% O₂) to evaluate the effects of low oxygen levels. Hypoxic conditions were created using an Invivo2 1000 hypoxia workstation (Ruskinn Technology, Pencoed, Wales) according to the manufacturers' instructions. The cultures were incubated at 37° C. in a humidified atmosphere containing 5% CO₂ and the cells were cultured with fresh complete medium. A half volume of medium was replaced twice a week. The whole adherent fraction was detached by trypsinization at 70-80% confluence (after 4-5 days) using Accutase (Chemicon Europe, Hampshire, UK) and re-plated. The FACS analysis, viability/apoptosis/necrosis study and cell cycle distribution analysis were performed after 10 days culture either in normoxia or in hypoxia and their potentiality was evaluated by their ability to differentiate towards adipogenic differentiation.

We found that hypoxia enhanced hAT-MSC proliferation: after ten days hypoxic cells were 18±1.4×10⁵ compared to 8.5±1×10⁵ in normoxia (n=4).

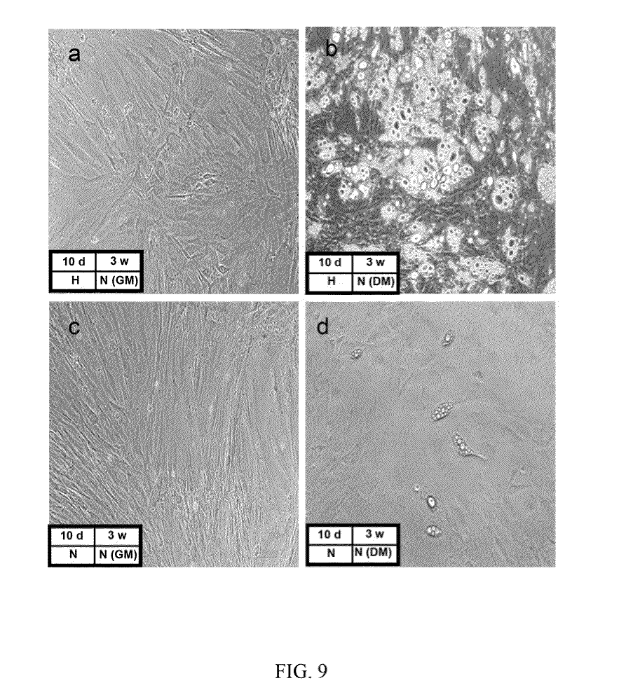

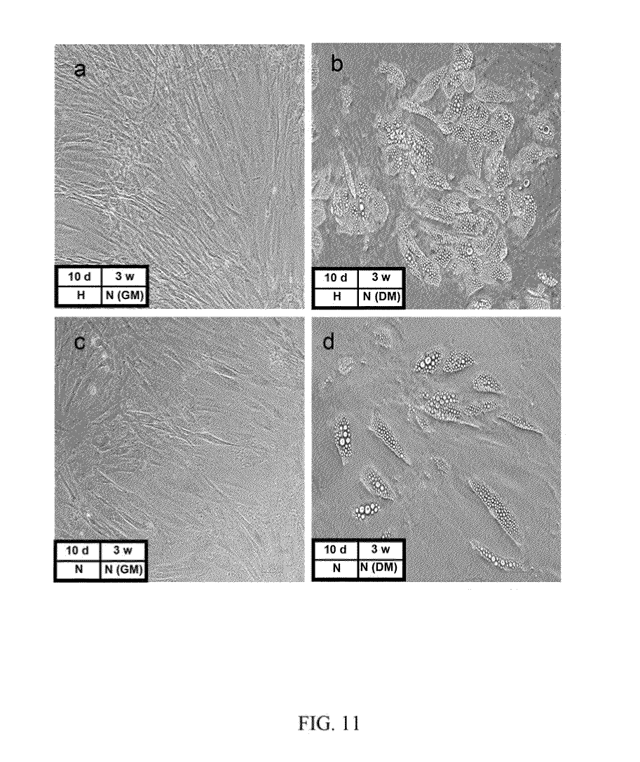

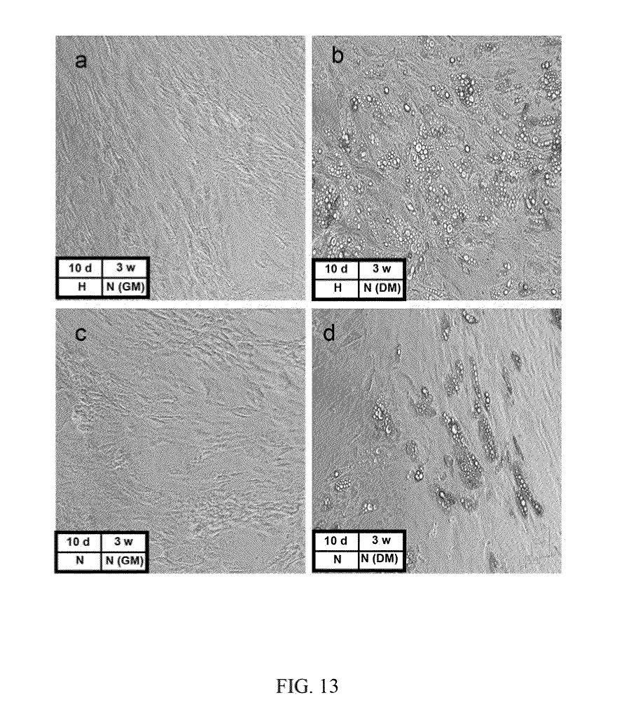

In order to evaluate the effect of pre-hypoxia exposure on adipogenic differentiation, hAT-MSCs were plated at 22×10³ cells/cm² corresponding to 50×10³ cells/well in 4 well plates in growth medium (GM) under normoxic or hypoxic conditions for 10 days. The cells were then transferred to normoxia and the GM was replaced with the adipogenic differentiation medium (DM). The hAT-MSCs were therefore cultured for 3 weeks in the presence of human MSC adipogenic induction medium (Lonza, Wokingham, UK). The differentiation culture medium was changed three times per week. Then, cells were fixed with 4% PFA in PBS for 20 min at room temperature, incubated in 60% iso-propyl-alcohol (IPA) and stained with 1% Oil Red O (Raymond Lamb, Eastbourne, UK) in IPA for 15 min and further incubated in IPA to remove background staining. Nuclei were stained with half-strength Harris' haematoxylin for 30 s, and then mounted in Glycergel. The positive fat vacuoles appeared as red-stained droplets. Oil Red-O staining performed after 3 weeks of differentiation showed that the cells pre exposed to hypoxia differentiated into adipocytes to a greater extent compared to the cells pre-exposed to normoxia (FIG. 9, FIG. 11, FIG. 13). In FIG. 9, Pre-hypoxic-cultured hAT-MSCs from the first donor display enhanced adipogenic differentiation potential when exposed to normoxia. hAT-MSCs were cultured under hypoxia in growth control medium (GM) for 10 days and transferred to normoxia in the presence of GM (FIG. 9a) or the adipogenic differentiation cocktail medium (DM) (FIG. 9b) for a period of 3 weeks. As controls, hAT-MSCs were cultured in normoxia and then exposed for the same time either to GM (FIG. 9c) or DM (FIG. 9d). Oil red O staining showed that lipid vacuoles accumulated to a greater extent in pre-hypoxic hAT-MSCs exposed to normoxic conditions under DM (FIG. 9b) compared to pre-normoxic hAT-MSCs cultured in DM (FIG. 9d). Bar=100 μm.

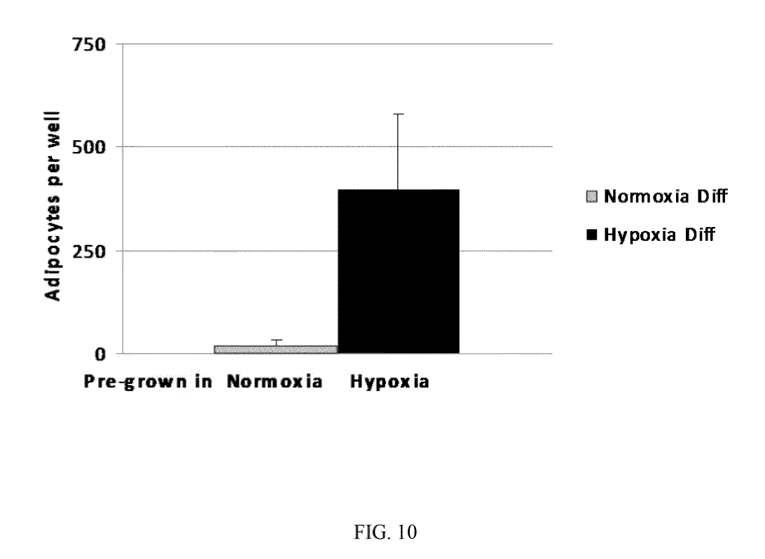

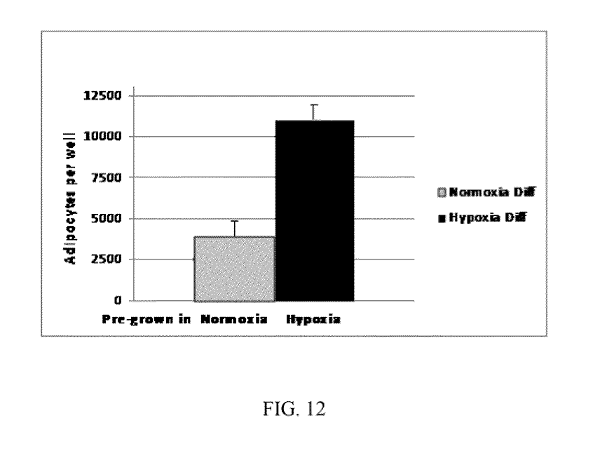

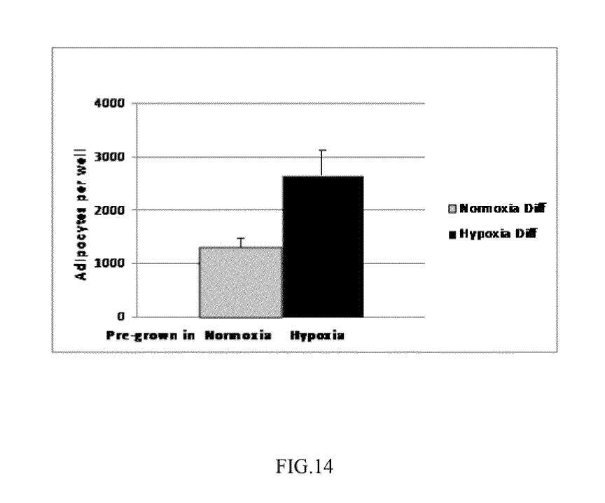

In order to evaluate the number of cells that have differentiated in adipocytes we have quantified the number of adipocytes on phase contrast microscope (n=4) (FIG. 10, FIG. 12, FIG. 14).

Therefore, although hypoxia inhibits adipogenic differentiation, pre-hypoxic-cultured hAT-MSCs demonstrated a higher adipogenic differentiation potential in normoxia compared to their prior normoxic-cultured counterparts.

All references cited in this specification are hereby incorporated by reference, each in its entirety. Any discussion of references cited herein is intended merely to summarize the assertions made by their authors and no admission is made that any reference or portion thereof constitutes relevant prior art. Applicants reserve the right to challenge the accuracy and pertinency of the cited references.

### REFERENCES

Challen, G. A. & Little, M. H. (2006) A side order of stem cells: the SP phenotype. Stem Cells, 24, 3-12.

Fink, T., Abildtrup, L., Fogd, K., Abdallah, B. M., Kassem, M., Ebbesen, P. & Zachar, V. (2004) Induction of adipocyte-like phenotype in human mesenchymal stem cells by hypoxia. Stem Cells, 22, 1346-1355.

Fleischaker, R. J., Weaver, J. C. & Sinskey, A. J. (1981) Instrumentation for process control in cell culture. Adv Appl Microbiol, 27, 137-167.

Gennaro, A. R. (1995) Remington: the science and practice of pharmacy, Mack, Easton, Pa.

Gnecchi M, He H, Noiseux N, Liang O D, Zhang L, Morello F, Mu H, Melo L G, Pratt R E, Ingwall J S, Dzau V J. Evidence supporting paracrine hypothesis for Akt-modified mesenchymal stem cell-mediated cardiac protection and functional improvement. FASEB J. 2006 April; 20(6):661-9.

Gomillion, C. T. & Burg, K. J. (2006) Stem cells and adipose tissue engineering. Biomaterials, 27, 6052-6063.

Gnecchi M, He H, Noiseux N, Liang O D, Zhang L, Morello F, Mu H, Melo L G, Pratt

Grayson et al, Hypoxia enhances proliferation and tissue formation of human mesenchymal stem cells, Biochem Biophys Res Commun, 358:948-953

Hardman, J. G., Limbird, L. E., Gilman, A. G., Goodman, L. S. & Gilman, A. (1996) Goodman & Gilman's the pharmacological basis of therapeutics, McGraw-Hill, Health Professions Division, New York; London.

Harlow, E. & Lane, D. (1999) Using antibodies: a laboratory manual, Cold Spring Harbour Laboratory Press, Cold Spring Harbour.

Hayashi, Y., Tsuji, S., Tsujii, M., Nishida, T., Ishii, S., Iijima, H., Nakamura, T., Eguchi, H., Miyoshi, E., Hayashi, N. & Kawano, S. (2008) Topical transplantation of mesenchymal stem cells accelerates gastric ulcer healing in rats. Am J Physiol Gastrointest Liver Physiol, 294, G778-786.

Hu, X., Yu, S. P., Fraser, J. L., Lu, Z., Ogle, M. E., Wang, J. A. & Wei, L. (2008) Transplantation of hypoxia-preconditioned mesenchymal stem cells improves infarcted heart function via enhanced survival of implanted cells and angiogenesis. J Thorac Cardiovasc Surg, 135, 799-808.

Hung, S. C., Pochampally, R. R., Hsu, S. C., Sanchez, C., Chen, S. C., Spees, J. & Prockop, D. J. (2007) Short-Term Exposure of Multipotent Stromal Cells to Low Oxygen Increases Their Expression of CX3CR1 and CXCR4 and Their Engraftment In Vivo. PLoS ONE, 2, e416.

Ivanovic, Z., Bartolozzi, B., Bernabei, P. A., Cipolleschi, M. G., Rovida, E., Milenkovic, P., Praloran, V. & Dello Sbarba, P. (2000) Incubation of murine bone marrow cells in hypoxia ensures the maintenance of marrow-repopulating ability together with the expansion of committed progenitors. Br J Haematol, 108, 424-429.

Ivanovic, Z., Dello Sbarba, P., Trimoreau, F., Faucher, J. L. & Praloran, V. (2000) Primitive human HPCs are better maintained and expanded in vitro at 1 percent oxygen than at 20 percent. Transfusion, 40, 1482-1488.

Kanichai, M., Ferguson, D., Prendergast, P. J. & Campbell, V. A. (2008) Hypoxia promotes chondrogenesis in rat mesenchymal stem cells: a role for AKT and hypoxia-inducible factor (HIF)-1alpha. J Cell Physiol, 216, 708-715.

Lanza, R. P. ed. (2004) Handbook of stem cells, Elsevier Academic Press, Amsterdam Boston.

Lanza, R. P. (2006) Essentials of stem cell biology, Elsevier Academic, Amsterdam; London.

Lo Cicero, V., Montelatici, E., Cantarella, G., Mazzola, R., Sambataro, G., Rebulla, P. & Lazzari, L. (2008) Do mesenchymal stem cells play a role in vocal fold fat graft survival? Cell Prolif, 41, 460-473.

Malladi, P., Xu, Y., Chiou, M., Giaccia, A. J. & Longaker, M. T. (2006) Effect of reduced oxygen tension on chondrogenesis and osteogenesis in adipose-derived mesenchymal cells. Am J Physiol Cell Physiol, 290, C1139-1146.

Minuth, W. W., Strehl, R. & Schumacher, K. (2005) Tissue engineering: essentials for daily laboratory work, Wiley-VCH, Weinheim; [Great Britain].

Orlic, D., Kajstura, J., Chimenti, S., Jakoniuk, I., Anderson, S. M., Li, B., Pickel, J., McKay, R., Nadal-Ginard, B., Bodine, D. M., Leri, A. & Anversa, P. (2001) Bone marrow cells regenerate infarcted myocardium. Nature, 410, 701-705.

Potier, E., Ferreira, E., Meunier, A., Sedel, L., Logeart-Avramoglou, D. & Petite, H. (2007) Prolonged hypoxia concomitant with serum deprivation induces massive human mesenchymal stem cell death. Tissue Eng, 13, 1325-1331.

Ren, H., Cao, Y., Zhao, Q., Li, J., Zhou, C., Liao, L., Jia, M., Zhao, Q., Cai, H., Han, Z. C., Yang, R., Chen, G. & Zhao, R. C. (2006) Proliferation and differentiation of bone marrow stromal cells under hypoxic conditions. Biochem Biophys Res Commun, 347, 12-21.

Riordan, N. H., Ichim, T. E., Min, W. P., Wang, H., Solano, F., Lara, F., Alfaro, M., Rodriguez, J. P., Harman, R. J., Patel, A. N., Murphy, M. P., Lee, R. R., and Minev, B. (2009) Non-expanded adipose stromal vascular fraction cell therapy for multiple sclerosis. Journal of Translational Medicine 7: 29.

Rowe, R. C., Sheskey, P. J. & Weller, P. J. (2003) Handbook of pharmaceutical excipients, Pharmaceutical Press, London.

Saltzman, W. M. (2004) Tissue engineering: engineering principles for the design of replacement organs and tissues, Oxford University Press, Oxford.

Sambrook, J. & Russell, D. W. (2001) Molecular cloning: a laboratory manual, Cold Spring Harbor Laboratory Press, Cold Spring Harbor, N.Y.

Spector, D. L., Goldman, R. D. & Leinwand, L. A. (1997) Cells: a laboratory manual, Cold Spring Harbor Laboratory Press, Cold Spring Harbor, N.Y.

Sterodimas, A., De Faria, J., Correa, W. E. & Pitanguy, I. (2009) Tissue engineering in plastic surgery: an up-to-date review of the current literature. Ann Plast Surg, 62, 97-103.

Stosich, M. S. & Mao, J. J. (2007) Adipose tissue engineering from human adult stem cells: clinical implications in plastic and reconstructive surgery. Plast Reconstr Surg. 119, 71-83; discussion 84-75.

Sung J H, Yang H M, Park J B, Choi G S, Joh J W, Kwon C H, Chun J M, Lee S K, Kim S J.(2008) Isolation and characterization of mouse mesenchymal stem cells. Transplant Proc. 40: 2649-2654.

Timper, K., Seboek, D., Eberhardt, M., Linscheid, P., Christ-Crain, M., Keller, U., Muller, B. & Zulewski, H. (2006) Human adipose tissue-derived mesenchymal stem cells differentiate into insulin, somatostatin, and glucagon expressing cells. Biochem Biophys Res Commun, 341, 1135-1140.

Vunjak-Novakovic and Freshney, eds. (2006) Culture of Cells for Tissue Engineering; Wiley-Liss, ISBN 0471629359

Vunjak-Novakovic, G. & Kaplan, D. L. (2006) Tissue engineering: the next generation. Tissue Eng, 12, 3261-3263.

Wilton, J., Jung, K., Vedin, I., Aronsson, B. & Flock, J. I. (1992) Comparative evaluation of a new molecular method for typing Staphylococcus epidermidis. Eur J Clin Microbiol Infect Dis, 11, 515-521.

Wu, Y., Chen, L., Scott, P. G. & Tredget, E. E. (2007) Mesenchymal stem cells enhance wound healing through differentiation and angiogenesis. Stem Cells, 25, 2648-2659.

Yoshikawa, T., Mitsuno, H., Nonaka, I., Sen, Y., Kawanishi, K., Inada, Y., Takakura, Y., Okuchi, K. & Nonomura, A. (2008) Wound therapy by marrow mesenchymal cell transplantation. Plast Reconstr Surg, 121, 860-877.

Aspects

The present disclosure includes the following aspects:

1. A method of forming an ex vivo cell culture comprising differentiated mesenchymal lineage cells, the method comprising:
a) providing a cell culture comprising a plurality of mesenchymal stem cells (MSCs);
b) subjecting the MSCs to hypoxic conditions; and
c) subsequent to b), subjecting the MSCs to normoxic conditions.

2. A method of forming an ex vivo cell culture in accordance with aspect 1, wherein the MSCs are adipose tissue MSCs (AT-MSCs).

3. A method of forming an ex vivo cell culture in accordance with aspect 2, wherein the AT-MSCs are epiploon AT-MSCs.

4. A method of forming an ex vivo cell culture in accordance with aspect 1, wherein the MSCs are bone marrow MSCs (BM-MSCs).

5. A method of forming an ex vivo cell culture in accordance with aspect 1, wherein the subjecting the MSCs to hypoxic conditions comprises subjecting the MSCs to an atmosphere comprising from about 0.2% oxygen up to about 7% oxygen.

6. A method of forming an ex vivo cell culture in accordance with aspect 1, wherein the subjecting the MSCs to hypoxic conditions comprises subjecting the MSCs to an atmosphere comprising from 0.2% oxygen up to 7% oxygen.

7. A method of forming an ex vivo cell culture in accordance with aspect 1, wherein the subjecting the MSCs to hypoxic conditions comprises subjecting the MSCs to an atmosphere comprising no more than about 2% oxygen.

8. A method of forming an ex vivo cell culture in accordance with aspect 1, wherein the subjecting the MSCs to hypoxic conditions comprises subjecting the MSCs to an atmosphere comprising no more than 2% oxygen.

9. A method of forming an ex vivo cell culture in accordance with aspect 1, wherein the subjecting the MSCs to hypoxic conditions comprises subjecting the MSCs to hypoxic conditions for from 1 day up to 14 days.

10. A method of forming an ex vivo cell culture in accordance with aspect I, wherein the subjecting the MSCs to hypoxic conditions comprises subjecting the MSCs to hypoxic conditions for from 3 days up to 14 days.

11. A method of forming an ex vivo cell culture in accordance with aspect 1, wherein the subjecting the MSCs to hypoxic conditions comprises subjecting the MSCs to hypoxic conditions for from 8 days up to 14 days.

12. A method of forming an ex vivo cell culture in accordance with aspect 1, wherein the subjecting the MSCs to hypoxic conditions comprises subjecting the MSCs to hypoxic conditions for 9 days up to 11 days.

13. A method of forming an ex vivo cell culture in accordance with aspect 1, wherein the subjecting the MSCs to hypoxic conditions comprises subjecting the MSCs to hypoxic conditions for about 10 days.

14. A method of forming an ex vivo cell culture in accordance with aspect 5, wherein the atmosphere further comprises about 5% CO2.

15. A method of forming an ex vivo cell culture in accordance with aspect 1, wherein the differentiated cells comprise adipocytes.

16. A method of forming an ex vivo cell culture in accordance with aspect 1, wherein the cell culture comprises at least 80% adipocyte lineage cells.

17. A method of forming an ex vivo cell culture in accordance with aspect I, wherein the differentiated cells comprise osteocytic lineage cells.

18. A method of forming an ex vivo cell culture in accordance with aspect 1, wherein the differentiated cells comprise chondrogenic lineage cells.

19. A method of forming an ex vivo cell culture in accordance with aspect 1, wherein the cell culture comprises an enhanced percentage of Oil Red O-staining-cells compared to a control culture exposed to normoxic conditions.

20. A method of forming an ex vivo cell culture in accordance with aspect 19, wherein the cell culture further comprises a medium comprising an effective amount of hydrocortisone, isobutyl methyl xantine, indomethacin, insulin or a combination thereof.

21. A method of forming an ex vivo cell culture in accordance with aspect 19, wherein the cell culture further comprises a medium comprising an effective amount of hydrocortisone, isobutyl methyl xantine, indomethacin and insulin.

22. A method of forming an ex vivo cell culture in accordance with aspect I, wherein the cell culture exposed to hypoxic conditions comprises an enhanced percentage of Alcian Blue-staining-cells compared to a control culture exposed to normoxic conditions.

23. A method of forming an ex vivo cell culture in accordance with aspect 22, wherein the cell culture further comprises a medium comprising an effective amount of basic Fibroblast Growth Factor (bFGF), Transforming Growth Factor-β1 (TGF β1), or a combination thereof.

24. A method of forming an ex vivo cell culture in accordance with aspect 22, wherein the cell culture further comprises a medium comprising an effective amount of basic Fibroblast Growth Factor (bFGF) and Transforming Growth Factor-β1 (TGF β1).

25. A method of forming an ex vivo cell culture in accordance with aspect 1, wherein the cell culture exposed to hypoxic conditions comprises an enhanced percentage of Von Kossa-staining-cells compared to a control culture not exposed to hypoxic conditions.

26. A method of forming an ex vivo cell culture in accordance with aspect 25, wherein the cell culture further comprises a medium comprising an effective amount of dexamethosone, vitamin C phosphate, sodium β-glycerophosphate, or a combination thereof.

27. A method of forming an ex vivo cell culture in accordance with aspect 25, wherein the cell culture further comprises a medium comprising an effective amount of dexamethosone, vitamin C phosphate, and sodium β-glycerophosphate.

26. A method of forming an ex vivo cell culture in accordance with aspect 1, wherein the ex vivo cell culture comprises adipose tissue.

27. A method of forming an ex vivo cell culture in accordance with aspect 1, wherein the ex vivo cell culture comprises ostocytic tissue.

28. A method of forming an ex vivo cell culture in accordance with aspect 1, wherein the ex vivo cell culture comprises chondrogenic tissue.

29. A method of repairing or augmenting a tissue or organ in a subject, comprising: forming an ex vivo cell culture in accordance with aspect 1; and transplanting cells comprised by the cell culture to the subject.

30. A method of repairing or augmenting a tissue or organ in a subject in accordance with aspect 29, wherein the cells are autologous to the subject.

31. A method of repairing or augmenting a tissue or organ in a subject in accordance with aspect 29, wherein the differentiated cells are selected from the group consisting of adipocyte lineage cells, osteocytic lineage cells, chondrogenic lineage cells and a combination thereof.

32. A method of repairing or augmenting a tissue or organ in a subject in accordance with aspect 29, wherein the tissue or organ in the subject is selected from the group consisting of bone, skin, breast and a combination thereof.

32. A method of repairing or augmenting a tissue or organ in a subject in accordance with aspect 29, wherein the tissue or organ is selected from the group consisting of breast, cheek, chin, lips, heart, and stomach.

33. A method of growing mesenchymal stem cells (MSCs) ex vivo, comprising:
providing a culture comprising MSCs; and
subjecting the culture to hypoxic conditions
   wherein the MSCs express at least one marker of MSC differentiation in an amount greater than that of a control culture comprising MSCs subjected to normoxic conditions.

34. A method of growing MSCs in accordance with aspect 33, wherein the at least one marker of MSC differentiation is selected from the group consisting of Sca1 and CD44.

35. A method of growing MSCs in accordance with aspect 33, wherein a greater percentage of the cells express Sca1 and CD44 compared to a control comprising MSCs subjected to normoxic conditions.

36. A method of growing MSCs in accordance with aspect 33, wherein the MSCs express the at least one marker of MSC differentiation in a greater percentage of cells compared to a control culture comprising MSCs subjected to normoxic conditions.

37. A method of growing MSCs in accordance with aspect 33, wherein the MSCs are adipose tissue MSCs (AT-MSCs).

38. A method of growing MSCs in accordance with aspect 33, wherein the MSCs are bone marrow MSCs (BM-MSCs).

39. A method of forming an ex vivo cell culture, comprising:
providing adipose tissue mesenchymal stem cells; and
growing the cells under hypoxic conditions,
   wherein cells comprising the cell culture ex vivo express one or more adipogenic markers at a level at least two-fold greater than a control cell culture that is subjected to normoxic conditions.

40. A method of forming an ex vivo cell culture in accordance with aspect 39, wherein the one or more adipocyte lineage differentiation markers are each selected from the group consisting of PPARγ, LPL and FBP4.

41. A method of increasing proliferation rate of a cell culture ex vivo, comprising growing the cells under hypoxic conditions, wherein the proliferation rate of the cell culture is greater than that of a control cell culture grown under normoxic conditions.

42. A method in accordance with aspect 41, wherein the cell culture comprises stem cells.

43. A method in accordance with aspect 42, wherein the stem cells are mesenchymal stem cells (MSCs).

44. A method in accordance with aspect 43, wherein the mesenchymal stem cells are adipose tissue mesenchymal stem cells (AT-MSCs).

45. A method in accordance with aspect 43, wherein the mesenchymal stem cells are bone marrow mesenchymal stem cells (BM-MSCs).

46. A method of enhancing expression of at least one pluripotent stem cell marker in an ex vivo cell culture, the method comprising:
a) providing a cell culture comprising a plurality of mesenchymal stem cells (MSCs); and
b) subjecting the MSCs to hypoxic conditions,
   wherein a greater percentage of cells express the at least one pluripotent stem cell marker compared to a cell culture comprising cells subjected to normoxic conditions.

47. A method of enhancing expression of at least one pluripotent stem cell marker in accordance with aspect 46, wherein the plurality of MSCs is a plurality of adipose tissue mesenchymal stem cells (AT-MSCs).

48. A method of enhancing expression of at least one pluripotent stem cell marker in accordance with aspect 46, wherein the plurality of MSCs is a plurality of bone marrow mesenchymal stem cells (BM-MSCs).

49. A method of enhancing expression of at least one pluripotent stem cell marker in accordance with aspect 46, wherein the at least one pleuripotent stem cell marker is selected from the group consisting of Sca1 and CD44.

50. A method of enhancing expression of at least one pluripotent stem cell marker in accordance with aspect 46, wherein greater than 35% of the MSCs are enriched in Sca1 and CD44.

51. A method of enhancing expression of at least one pluripotent stem cell marker in accordance with aspect 47, wherein greater than 35% up to about 80% of the AT-MSCs are enriched in Sca1 and CD44.

52. A method of maintaining mesenchymal stem cells in an undifferentiated state, the method comprising maintaining the mesenchymal stem cells under hypoxic conditions ex vivo.

53. A method of maintaining mesenchymal stem cells (MSCs) in an undifferentiated state in accordance with aspect 51, wherein the maintaining the mesenchymal stem cells under hypoxic conditions comprises maintaining the cells in an atmosphere comprising from 1% to 10% oxygen.

54. A method of maintaining mesenchymal stem cells (MSCs) in an undifferentiated state in accordance with aspect 51, wherein the maintaining the mesenchymal stem cells under hypoxic conditions comprises maintaining the cells in an atmosphere comprising from 0.2% to 3% oxygen.

55. A method of maintaining mesenchymal stem cells (MSCs) in an undifferentiated state in accordance with aspect 51, wherein the maintaining the mesenchymal stem cells under hypoxic conditions comprises maintaining the cells in an atmosphere comprising about 2% oxygen.

56. A method of enhancing expression of at least one adipogenic lineage gene in an ex vivo cell culture, the method comprising:
providing an ex vivo cell culture comprising mesenchymal stem cells (MSCs);
growing the cells under hypoxic conditions; and
returning the cells to normoxic conditions,
   whereby the at least one adipogenic lineage genes is expressed at a level greater than that of a control culture grown under normoxic conditions.

57. A method of enhancing expression of at least one adipogenic lineage gene in an ex vivo cell culture in accordance with aspect 56, wherein the MSCs are adipose tissue MSCs (AT-MSCs).

58. A method of enhancing expression of one or more adipogenic lineage genes in an ex vivo cell culture in accordance with aspect 56, wherein the adipogenic lineage genes are selected from the group consisting of PPARγ, LPL and FABP.

59. A method of promoting healing of a gastric ulcer, comprising:
forming an ex vivo cell culture comprising differentiated adipose tissue MSCs in accordance with the method of aspect 1, wherein the subjecting the MSCs to normoxic conditions comprises subjecting the MSCs to normoxia under conditions that promote expression of mRNAs for VEGF and hepatocyte growth factor (HGF); and
transplanting the cells to gastric tissue surrounding the ulcer in a subject in need of treatment. Hayashi, Y. et al., Am J Physiol Gastrointest Liver Physiol 294: G778-G786, 2008.

60. A method of promoting heart regeneration in a subject, comprising:
forming an ex vivo cell culture comprising differentiated adipose tissue mesenchymal stem cells (AT-MSCs) in accordance with the method of aspect 1, wherein the subjecting the MSCs to normoxic conditions comprises subjecting the MSCs to normoxia under conditions that promote increased expression of pro-survival and pro-angiogenic factors; and
transplanting the cells to a diseased area of the heart in a subject in need of treatment.

61. A method of promoting wound healing in a subject, comprising:
forming an ex vivo cell culture comprising differentiated adipose tissue mesenchymal stem cells (AT-MSCs) in accordance with the method of aspect 1, wherein the subjecting the MSCs to normoxic conditions comprises subjecting the MSCs to normoxia under conditions that promote increased expression and release of proangiogenic factors; and
transplanting the cells to a diseased area for cutaneous regeneration or wound healing in a subject in need of treatment.

Wu, Y., et al., Stem Cells 25: 2648-2659, 2007

62. A method of promoting repair or regeneration of a tissue in a subject, comprising:
forming an ex vivo cell culture comprising differentiated adipose tissue mesenchymal stem cells (AT-MSCs) in accordance with the method of aspect 1, wherein the subjecting the MSCs to normoxic conditions comprises subjecting the MSCs to normoxia under conditions that promote increased expression of pro-survival and pro-angiogenic factors; and
transplanting the cells to a diseased area of the tissue in a subject in need of treatment. Hu, X., et al., J. Thorac. Cariovasc. Surg. 135: 799-808, 2008.

63. A method of promoting repair or regeneration of a tissue in accordance with aspect 62, wherein the tissue is selected from the group consisting of breast, cheek, chin and lip.

64. An ex vivo cell culture comprising mesenchymal stem cells differentiated as adipose lineage cells at a greater percentage compared to a control ex vivo cell culture comprising adipose tissue mesenchymal stem cells grown under normoxic conditions.

65. An ex vivo cell culture in accordance with aspect 64, wherein the adipose lineage cells are selected from the group consisting of adipocytes, osteocytes, chondrocytes and a combination thereof.

66. An ex vivo cell culture in accordance with aspect 64, wherein the culture comprises a plurality of adipocytes.

67. An ex vivo cell culture in accordance with aspect 66, wherein the culture further comprises hydrocortisone, isobutyl xanthine, indomethacin and insulin.

68. An ex vivo cell culture in accordance with aspect 63, wherein the culture comprises a plurality of chondrocytes.

69. An ex vivo cell culture in accordance with aspect 68, wherein the culture further comprises basic Fibroblast Growth Factor and Transforming Growth Factor-β1.

70. An ex vivo cell culture in accordance with aspect 63, wherein the culture comprises a plurality of osteocytes.

71. An ex vivo cell culture in accordance with aspect 70, wherein the culture further comprises dexamethasone, vitamin C phosphate, and sodium-β-glycerophosphate.

72. A method in accordance with any one of aspects 1-63, wherein the cell culture consists of human cells.

73. A method in accordance with any one of aspects 1-63, wherein the cell culture comprises human cells.

74. An ex vivo cell culture in accordance with any one of aspects 64-71, wherein the cell culture consists of human cells.

74. An ex vivo cell culture in accordance with any one of aspects 64-71, wherein the cell culture comprises human cells.

What is claimed is:

1. A method of forming an ex vivo cell culture comprising differentiated mesenchymal lineage cells, the method comprising:
a) providing a cell culture comprising a plurality of mesenchymal stem cells (MSCs);
b) subjecting the MSCs to hypoxic conditions; and
c) subsequent to b), subjecting the MSCs to normoxic conditions.

2. A method of forming an ex vivo cell culture in accordance with claim 1, wherein the MSCs are mammalian MSCs.

3. A method of forming an ex vivo cell culture in accordance with claim 1, wherein the MSCs are murine MSCs.

4. A method of forming an ex vivo cell culture in accordance with claim 1, wherein the MSCs are human MSCs.

5. A method of forming an ex vivo cell culture in accordance with claim 1, wherein the MSCs are adipose tissue MSCs (AT-MSCs).

6. A method of forming an ex vivo cell culture in accordance with claim 5, wherein the AT-MSCs are epiploon AT-MSCs.

7. A method of forming an ex vivo cell culture in accordance with claim 1, wherein the MSCs are bone marrow MSCs (BM-MSCs).

8. A method of forming an ex vivo cell culture in accordance with claim 1, wherein the subjecting the MSCs to hypoxic conditions comprises subjecting the MSCs to an atmosphere comprising from about 0.2% oxygen up to about 7% oxygen.

9. A method of forming an ex vivo cell culture in accordance with claim 1, wherein the subjecting the MSCs to hypoxic conditions comprises subjecting the MSCs to an atmosphere comprising from 0.2% oxygen up to 7% oxygen.

10. A method of forming an ex vivo cell culture in accordance with claim 1, wherein the subjecting the MSCs to hypoxic conditions comprises subjecting the MSCs to an atmosphere comprising no more than about 2% oxygen.

11. A method of forming an ex vivo cell culture in accordance with claim 1, wherein the subjecting the MSCs to hypoxic conditions comprises subjecting the MSCs to an atmosphere comprising no more than 2% oxygen.

12. A method of forming an ex vivo cell culture in accordance with claim 1, wherein the subjecting the MSCs to hypoxic conditions comprises subjecting the MSCs to hypoxic conditions for from 1 day up to 14 days.

13. A method of forming an ex vivo cell culture in accordance with claim 1, wherein the subjecting the MSCs to hypoxic conditions comprises subjecting the MSCs to hypoxic conditions for from 3 days up to 14 days.

14. A method of forming an ex vivo cell culture in accordance with claim 1, wherein the subjecting the MSCs to hypoxic conditions comprises subjecting the MSCs to hypoxic conditions for from 8 days up to 14 days.

15. A method of forming an ex vivo cell culture in accordance with claim 1, wherein the subjecting the MSCs to hypoxic conditions comprises subjecting the MSCs to hypoxic conditions for 9 days up to 11 days.

16. A method of forming an ex vivo cell culture in accordance with claim 1, wherein the subjecting the MSCs to hypoxic conditions comprises subjecting the MSCs to hypoxic conditions for about 10 days.

17. A method of forming an ex vivo cell culture in accordance with claim 5, wherein the atmosphere further comprises about 5% CO₂.

18. A method of forming an ex vivo cell culture in accordance with claim 1, wherein the differentiated cells comprise adipocytes.

19. A method of forming an ex vivo cell culture in accordance with claim 1, wherein the cell culture comprises at least 80% adipocyte lineage cells.

20. A method of forming an ex vivo cell culture in accordance with claim 1, wherein the differentiated cells comprise osteocytic lineage cells.

21. A method of forming an ex vivo cell culture in accordance with claim 1, wherein the differentiated cells comprise chondrogenic lineage cells.

22. A method of forming an ex vivo cell culture in accordance with claim 1, wherein the cell culture comprises an enhanced percentage of Oil Red O-staining-cells compared to a control culture exposed to normoxic conditions.

23. A method of forming an ex vivo cell culture in accordance with claim 22, wherein the cell culture further comprises a medium comprising an effective amount of hydrocortisone, isobutyl methyl xantine, indomethacin, insulin or a combination thereof.

24. A method of forming an ex vivo cell culture in accordance with claim 22, wherein the cell culture further comprises a medium comprising an effective amount of hydrocortisone, isobutyl methyl xantine, indomethacin and insulin.

25. A method of forming an ex vivo cell culture in accordance with claim 1, wherein the cell culture exposed to hypoxic conditions comprises an enhanced percentage of Alcian Blue-staining-cells compared to a control culture exposed to normoxic conditions.

26. A method of forming an ex vivo cell culture in accordance with claim 25, wherein the cell culture further comprises a medium comprising an effective amount of basic Fibroblast Growth Factor (bFGF), Transforming Growth Factor-β1 (TGF β1), or a combination thereof.

27. A method of forming an ex vivo cell culture in accordance with claim 24, wherein the cell culture further comprises a medium comprising an effective amount of basic Fibroblast Growth Factor (bFGF) and Transforming Growth Factor-β1 (TGF β1).

28. A method of forming an ex vivo cell culture in accordance with claim 1, wherein the cell culture exposed to hypoxic conditions comprises an enhanced percentage of Von Kossa-staining-cells compared to a control culture not exposed to hypoxic conditions.

29. A method of forming an ex vivo cell culture in accordance with claim 28, wherein the cell culture further comprises a medium comprising an effective amount of dexamethosone, vitamin C phosphate, sodium β-glycerophosphate, or a combination thereof.

30. A method of forming an ex vivo cell culture in accordance with claim 28, wherein the cell culture further comprises a medium comprising an effective amount of dexamethosone, vitamin C phosphate, and sodium β-glycerophosphate.

31. A method of forming an ex vivo cell culture in accordance with claim 1, wherein the ex vivo cell culture comprises adipose tissue.

32. A method of forming an ex vivo cell culture in accordance with claim 1, wherein the ex vivo cell culture comprises ostocytic tissue.

33. A method of forming an ex vivo cell culture in accordance with claim 1, wherein the ex vivo cell culture comprises chondrogenic tissue.

34. A method of repairing or augmenting a tissue or organ in a subject, comprising:
forming an ex vivo cell culture in accordance with claim 1; and
transplanting cells comprised by the cell culture to the subject.

35. A method of repairing or augmenting a tissue or organ in a subject in accordance with claim 34, wherein the cells are autologous to the subject.

36. A method of repairing or augmenting a tissue or organ in a subject in accordance with claim 34, wherein the differentiated cells are selected from the group consisting of adipocyte lineage cells, osteocytic lineage cells, chondrogenic lineage cells and a combination thereof.

37. A method of repairing or augmenting a tissue or organ in a subject in accordance with claim 34, wherein the tissue or organ in the subject is selected from the group consisting of bone, skin, breast and a combination thereof.

38. A method of repairing or augmenting a tissue or organ in a subject in accordance with claim 34, wherein the tissue or organ is selected from the group consisting of breast, cheek, chin, lips, heart, and stomach.

39. A method of growing mesenchymal stem cells (MSCs) ex vivo, comprising:
providing a culture comprising MSCs; and
subjecting the culture to hypoxic conditions
wherein the MSCs express at least one marker of MSC differentiation in an amount greater than that of a control culture comprising MSCs subjected to normoxic conditions.

40. A method of growing MSCs in accordance with claim 39, wherein the MSCs are murine MSCs.

41. A method of growing MSCs in accordance with claim 39, wherein the MSCs are human MSCs.

42. A method of growing MSCs in accordance with claim 39, wherein the at least one marker of MSC differentiation is selected from the group consisting of Sca1 and CD44.

43. A method of growing MSCs in accordance with claim 39, wherein a greater percentage of the cells express Sca1 and CD44 compared to a control comprising MSCs subjected to normoxic conditions.

44. A method of growing MSCs in accordance with claim 39, wherein the MSCs express the at least one marker of MSC differentiation in a greater percentage of cells compared to a control culture comprising MSCs subjected to normoxic conditions.

45. A method of growing MSCs in accordance with claim 39, wherein the MSCs are adipose tissue MSCs (AT-MSCs).

46. A method of growing MSCs in accordance with claim 39, wherein the MSCs are bone marrow MSCs (BM-MSCs).

47. A method of forming an ex vivo cell culture, comprising:
providing adipose tissue mesenchymal stem cells; and
growing the cells under hypoxic conditions,
wherein cells comprising the cell culture ex vivo express one or more adipogenic markers at a level at least two-fold greater than a control cell culture that is subjected to normoxic conditions.

48. A method of forming an ex vivo cell culture in accordance with claim 47, wherein the cells are murine cells.

49. A method of forming an ex vivo cell culture in accordance with claim 47, wherein the cells are human cells.

50. A method of forming an ex vivo cell culture in accordance with claim 47, wherein the one or more adipocyte lineage differentiation markers are each selected from the group consisting of PPARγ, LPL and FBP4.

51. A method of increasing proliferation rate of a cell culture ex vivo, comprising growing the cells under hypoxic conditions, wherein the proliferation rate of the cell culture is greater than that of a control cell culture grown under normoxic conditions.

52. A method in accordance with claim 51, wherein the cell culture comprises stem cells.

53. A method in accordance with claim 67, wherein the cells are mammalian cells.

54. A method in accordance with claim 67, wherein the cells are murine cells.

55. A method in accordance with claim 67, wherein the cells are human cells.

56. A method in accordance with claim 67, wherein the stem cells are mesenchymal stem cells (MSCs).

57. A method in accordance with claim 56, wherein the mesenchymal stem cells are adipose tissue mesenchymal stem cells (AT-MSCs).

58. A method in accordance with claim 56, wherein the mesenchymal stem cells are bone marrow mesenchymal stein cells (BM-MSCs).

59. A method of enhancing expression of at least one pluripotent stem cell marker in an ex vivo cell culture, the method comprising:
a) providing a cell culture comprising a plurality of mesenchymal stein cells (MSCs); and
b) subjecting the MSCs to hypoxic conditions,
wherein a greater percentage of cells express the at least one pluripotent stem cell marker compared to a cell culture comprising cells subjected to normoxic conditions.

60. A method of enhancing expression of at least one pluripotent stem cell marker in accordance with claim 59, wherein the plurality of MSCs is a plurality of murine MSCs.

61. A method of enhancing expression of at least one pluripotent stem cell marker in accordance with claim 59, wherein the plurality of MSCs is a plurality of human MSCs.

62. A method of enhancing expression of at least one pluripotent stem cell marker in accordance with claim 59, wherein the plurality of MSCs is a plurality of adipose tissue mesenchymal stem cells (AT-MSCs).

63. A method of enhancing expression of at least one pluripotent stem cell marker in accordance with claim 59, wherein the plurality of MSCs is a plurality of bone marrow mesenchymal stem cells (BM-MSCs).

64. A method of enhancing expression of at least one pluripotent stem cell marker in accordance with claim 59, wherein the at least one pleuripotent stem cell marker is selected from the group consisting of Sca1 and CD44.

65. A method of enhancing expression of at least one pluripotent stem cell marker in accordance with claim 59, wherein greater than 35% of the MSCs are enriched in Sca1 and CD44.

66. A method of enhancing expression of at least one pluripotent stem cell marker in accordance with claim 59, wherein greater than 35% up to about 80% of the AT-MSCs are enriched in Sca1 and CD44.

67. A method of maintaining mesenchymal stem cells in an undifferentiated state, the method comprising maintaining the mesenchymal stem cells under hypoxic conditions ex vivo.

68. A method of maintaining mesenchymal stem cells (MSCs) in an undifferentiated state in accordance with claim 67, wherein the mesenchymals stems cells are murine mesenchymal stem cells.

69. A method of maintaining mesenchymal stem cells (MSCs) in an undifferentiated state in accordance with claim 67, wherein the mesenchymals stems cells are human mesenchymal stem cells.

70. A method of maintaining mesenchymal stem cells (MSCs) in an undifferentiated state in accordance with claim 67, wherein the maintaining the mesenchymal stem cells under hypoxic conditions comprises maintaining the cells in an atmosphere comprising from 1% to 10% oxygen.

71. A method of maintaining mesenchymal stem cells (MSCs) in an undifferentiated state in accordance with claim 67, wherein the maintaining the mesenchymal stem cells under hypoxic conditions comprises maintaining the cells in an atmosphere comprising from 0.2% to 3% oxygen.

72. A method of maintaining mesenchymal stem cells (MSCs) in an undifferentiated state in accordance with claim 67, wherein the maintaining the mesenchymal stem cells under hypoxic conditions comprises maintaining the cells in an atmosphere comprising about 2% oxygen.

73. A method of enhancing expression of at least one adipogenic lineage gene in an ex vivo cell culture, the method comprising:
providing an ex vivo cell culture comprising mesenchymal stem cells (MSCs);
growing the cells under hypoxic conditions; and
returning the cells to normoxic conditions,
whereby the at least one adipogenic lineage genes is expressed at a level greater than that of a control culture grown under normoxic conditions.

74. A method of enhancing expression of at least one adipogenic lineage gene in an ex vivo cell culture in accordance with claim 73, wherein the MSCs are murine MSCs.

75. A method of enhancing expression of at least one adipogenic lineage gene in an ex vivo cell culture in accordance with claim 73, wherein the MSCs are murine MSCs.

76. A method of enhancing expression of at least one adipogenic lineage gene in an ex vivo cell culture in accordance with claim 73, wherein the MSCs are adipose tissue MSCs (AT-MSCs).

77. A method of enhancing expression of one or more adipogenic lineage genes in an ex vivo cell culture in accordance with claim 73, wherein the adipogenic lineage genes are selected from the group consisting of PPARγ, LPL and FABP.

78. A method of promoting healing of a gastric ulcer, comprising:
forming an ex vivo cell culture comprising differentiated adipose tissue MSCs in accordance with the method of claim 1, wherein the subjecting the MSCs to normoxic conditions comprises subjecting the MSCs to normoxia under conditions that promote expression of mRNAs for VEGF and hepatocyte growth factor (HGF); and
transplanting the cells to gastric tissue surrounding the ulcer in a subject in need of treatment.

79. A method of promoting heart regeneration in a subject, comprising:
forming an ex vivo cell culture comprising differentiated adipose tissue mesenchymal stem cells (AT-MSCs) in accordance with the method of claim 1, wherein the subjecting the MSCs to normoxic conditions comprises subjecting the MSCs to normoxia under conditions that promote increased expression of pro-survival and pro-angiogenic factors; and
transplanting the cells to a diseased area of the heart in a subject in need of treatment.

80. A method of promoting wound healing in a subject, comprising:
forming an ex vivo cell culture comprising differentiated adipose tissue mesenchymal stem cells (AT-MSCs) in accordance with the method of claim 1, wherein the subjecting the MSCs to normoxic conditions comprises subjecting the MSCs to normoxia under conditions that promote increased expression and release of proangiogenic factors; and
transplanting the cells to a diseased area for cutaneous regeneration or wound healing in a subject in need of treatment.

81. A method of promoting repair or regeneration of a tissue in a subject, comprising:
forming an ex vivo cell culture comprising differentiated adipose tissue mesenchymal stem cells (AT-MSCs) in accordance with the method of claim 1, wherein the subjecting the MSCs to normoxic conditions comprises subjecting the MSCs to normoxia under conditions that promote increased expression of pro-survival and pro-angiogenic factors; and
transplanting the cells to a diseased area of the tissue in a subject in need of treatment.

82. A method of promoting repair or regeneration of a tissue in accordance with claim 62, wherein the tissue is selected from the group consisting of breast, cheek, chin and lip.

83. An ex vivo cell culture comprising mesenchymal stem cells differentiated as adipose lineage cells at a greater percentage compared to a control ex vivo cell culture comprising adipose tissue mesenchymal stem cells grown under normoxic conditions.

84. An ex vivo cell culture in accordance with claim 83, wherein the mesenchymal stem cells are murine mesenchymal stem cells.

85. An ex vivo cell culture in accordance with claim 83, wherein the mesenchymal stem cells are human mesenchymal stem cells.

86. An ex vivo cell culture in accordance with claim 83, wherein the adipose lineage cells are selected from the group consisting of adipocytes, osteocytes, chondrocytes and a combination thereof.

87. An ex vivo cell culture in accordance with claim 83, wherein the culture comprises a plurality of adipocytes.

88. An ex vivo cell culture in accordance with claim 87, wherein the culture further comprises hydrocortisone, isobutyl xanthine, indomethacin and insulin.

89. An ex vivo cell culture in accordance with claim 83, wherein the culture comprises a plurality of chondrocytes.

90. An ex vivo cell culture in accordance with claim 83, wherein the culture further comprises basic Fibroblast Growth Factor and Transforming Growth Factor-β1.

91. An ex vivo cell culture in accordance with claim 83, wherein the culture comprises a plurality of osteocytes.

92. An ex vivo cell culture in accordance with claim 91, wherein the culture further comprises dexamethasone, vitamin C phosphate, and sodium-β-glycerophosphate.
